(19) United States
(12) Patent Application Publication
(10) Pub. No.: US 2021/0022305 A1
(43) Pub. Date: Jan. 28, 2021

(54) A HUMULUS PLANT VARIANT AND EXTRACTS THEREOF

(71) Applicant: BOMI LLC, Reno, NV (US)

(72) Inventor: Bomi JOSEPH, Los Gatos, CA (US)

(21) Appl. No.: 16/980,655

(22) PCT Filed: Mar. 12, 2019
(86) PCT No.: PCT/US2019/021857

§ 371 Date: Sep. 14, 2020

Related U.S. Application Data
(60) Provisional application No. 62/641,778, filed on Mar. 12, 2018.

(51) Int. Cl. A01H 6/28; A61K 36/185; A61K 31/015; A61K 31/05; A61K 9/20; A61K 9/00; A01H 5/12

(52) U.S. Cl. CPC A01H 6/28; A61K 36/185; A61K 31/015; A01H 5/12; A61K 9/20; A61K 9/0014; A61K 31/05

(57) ABSTRACT

The present invention is directed to a Humulus plant having high cannabinoid levels in it leaves and inflorescence. The present invention is also directed to extracts of this Humulus plant, compositions comprising the Humulus plant extracts, and methods of treatment involving these compositions.

This application claims the benefit to U.S. Provisional Patent Application Ser. No. 62/641,778, filed on Mar. 12, 2018, which is hereby incorporated by reference in its entirety.

### FIELD OF THE INVENTION

The present invention is directed to a Humulus plant having high cannabinoid levels in it leaves and inflorescence. The present invention is also directed to extracts of this Humulus plant, compositions comprising the Humulus plant extracts, and methods of treatment involving these compositions.

### BACKGROUND OF THE INVENTION

Cannabidiol (CBD) is one of many active cannabinoids identified in Cannabis. It is a major phytocannabinoid and accounts for up to 40% of the plant's extract. While initially considered an inactive cannabinoid, it is now well recognized that CBD possesses a wider scope of pharmacological properties than tetrahydrocannabinol (THC), and lacks the psychoactive effects. For example, CBD suppresses inflammation and neuropathic pain (Xiong et al., “Cannabinoids Suppress Inflammatory and Neuropathic Pain by Targeting alpha3 Glycine Receptors,” J. Exp. Med. 209(6): 1121-1134 (2012)), reduces anxiety and stress (Crippa et al., “Neural Basis of Anxiolytic Effects of Cannabidiol (CBD) in Generalized Social Anxiety Disorder: A Preliminary Report,” J. Psychopharmacol. 25(1): 121-130 (2011); Hill et al., “Endogenous Cannabinoid Signaling is Essential for Stress Adaption,” Proc. Nat'l Acad. Sci. USA 107(20): 9406-11 (2010)), possesses antiemetic properties (Sharkey et al., “Regulation of Nausea and Vomiting by Cannabinoids and the Endocannabinoid System,” Eur. J. Pharmacol. 722:134-46 (2014)), anticonvulsant properties (Karler and Turkanis, “The Cannabinoids as Potential Antiepileptics,” J. Clin. Pharmacol. 21(8-9 Suppl): 437S-448S (1981)), antipsychotic properties (Schubart et al., “Cannabidiol as a Potential Treatment for Psychosis,” Eur. Neuropsychopharmacol. 24(1): 51-64 (2014)), anti-inflammatory properties (Burstein and Zurier, “Cannabinoids, Endocannabinoids, and Related Analogs in Inflammation,” AAPS J. 11(1): 109-19 (2009)), anti-tumoral properties (Massi et al., “Cannabidiol as Potential Anticancer Drug,” Br. J. Clin. Pharmacol. 75(2): 303-12 (2013)), anxiolytic properties (Schier et al., “Cannabidiol, a Cannabis sativa Constituent, as an Anxiolytic Drug,” Rev. Bras Psiquiatr. 34(Suppl 1): S104-10 (2012)), and anti-depressant properties (Zanelati et al., “Antidepressant-like Effects of Cannabidiol in Mice: Possible Involvement of 5-HT1A Receptors,” Br. J. Pharmacol. 159(1): 122-28 (2010)). CBD also exerts potent anti-bacterial activity (Klingeren and Ten Ham, “Antibacterial Activity of Delta9-tetrahydrocannabinol and Cannabidiol,” Antonie Van Leeuwenhoek 42(1-2): 9-12 (1976) and Appendino et al., “Antibacterial Cannabinoids from Cannabis sativa: A Structure-Activity Study,” J. Nat. Prod. 71(8): 1427-30 (2008)).

In order to fully exploit the pharmacological potential of CBD it is essential to have highly pure cannabidiol preparations obtainable using methods that are easy, relatively inexpensive, and capable of scale-up. However, since the primary source of CBD is the Cannabis plant, and methods of extraction are not selective to CBD, the resulting extracts often include significant and restrictive amounts of psychoactive cannabinoids such as THC.

While synthetic forms of cannabidiol are available, these forms are expensive and lack the bioactivity of the naturally occurring plant derived forms of CBD. Accordingly, there is a need in the art to provide a source of bioactive CBD, i.e., natural CBD, that is free of psychoactive cannabinoids like THC.

The present invention overcomes this and other deficiencies in the art.

### SUMMARY OF THE INVENTION

One aspect of the present invention is directed to a Humulus plant having a concentration of cannabinoids in its inflorescence of at least 75 mg/g (dry weight).

Another aspect of the present invention is directed to an extract of the Humulus plant as described herein.

Another aspect of the present invention is directed to an extract of the Humulus plant comprising concentrated levels of humulene, β-caryophyllene, and cannabidiol.

Another aspect of the present invention is directed to a composition comprising the Humulus plant extract. This composition comprises humulene, β-caryophyllene, and cannabidiol.

Another aspect of the present invention is directed to a method of modulating endocannabinoid system activity in a subject. This method involves selecting a subject in need of endocannabinoid system modulation, and administering to the selected subject, the Humulus plant extract as described herein, or a composition comprising the Humulus plant extract in an amount effective to modulate endocannabinoid system activity in the subject.

### BRIEF DESCRIPTION OF THE DRAWINGS

FIG. 1 is a photograph of the Humulus kriya plant growing in a greenhouse in Ooty, India in July 2017.

FIG. 2A shows the Humulus yunnanensis var kriya leaf.

FIGS. 3A-3B are photographs of the Humulus yunnanensis var kriya inflorescence at different stages. FIG. 3A is a picture of a ‘kriya’ inflorescence during the mid-vegetative growth stage. FIG. 3B is a picture of a ‘kriya’ inflorescence during the late reproductive stage.

FIG. 4 shows reduction in calcification of VIC cells. Across all five mg concentrations, there was greater calcification reduction amidst exposure to higher bioactivity cannabidiol (CBD). Variance increased as reduction proportions came closer to 0.5.

FIG. 5 shows reduction in calcification of VIC cells. When bioactivity was very low, concentration made a negligible, inconsistent difference (see FIG. 5, top graph). When bioactivity was very high, higher concentrations yielded greater calcification reductions (see FIG. 5, bottom graph).

FIG. 6 shows treatment of VIC cells with ImmunAG, a composition comprising, CBD, β-caryophyllene, and humulene, reduces calcification significantly more than treatment with CBD alone across all doses.

FIGS. 7A-7B show changes in tumor size following cisplatin only treatment. FIG. 7A shows the initial tumor sizes for cisplatin only treatment. Sample Size, n: 172. Mean: 4.85116. Median: 4.83. Midrange: 4.74. RMS: 4.872. Variance, s\hat{ }2: 0.2038. Standard Deviation, s: 0.45145. Mean Absolute Deviation: 0.37177. Range: 2.4200000000000004. Coefficient of Variance: 9.30593%. Minimum: 3.53. 1st Quartile: 4.53. 2nd Quartile: 4.83. 3rd Quartile: 5.175. Maximum: 5.95. Sum: 834.4. Sum of Squares: 4082.6606. 95% CI for the Mean: 4.78322<mean<4.91911. 95% CI for the Standard Deviation: 0.40825<SD<0.50495. 95% CI for the Variance: 0.16667<VAR<0.25497. FIG. 7B shows the final tumor sizes for cisplatin only treatment. Sample Size, n: 172. Mean: 5.65946. Median: 5.596. Midrange: 5.567. RMS: 5.86443. Variance, s\hat{ }2: 2.37592. Standard Deviation, s: 1.5414. Mean Absolute Deviation: 1.32113. Range: 6.408000000000001. Coefficient of Variance: 27.23585%. Minimum: 2.363. 1st Quartile: 4.38. 2nd Quartile: 5.596. 3rd Quartile: 6.9065. Maximum: 8.771. Sum: 973.427. Sum of Squares: 5915.35269. 95% CI for the Mean: 5.42746<mean<5.89146. 95% CI for the Standard Deviation: 1.39391<SD<1.72407. 95% CI for the Variance: 1.94299<VAR<2.97242

FIGS. 8A-8B show changes in tumor sizes following combination treatment with cisplatin and ImmunAG as adjuvant treatment. FIG. 8A shows initial tumor sizes for cisplatin with ImmunAG as adjuvant treatment. Sample Size, n: 138. Mean: 4.88587. Median: 4.855. Midrange: 5.06. RMS: 4.90409. Variance, s\hat{ }2: 0.17971. Standard Deviation, s: 0.42393. Mean Absolute Deviation: 0.33102. Range: 2.18. Coefficient of Variance: 8.67658%. Minimum: 3.97. 1st Quartile: 4.62. 2nd Quartile: 4.855. 3rd Quartile: 5.11. Maximum: 6.15. Sum: 674.25. Sum of Squares: 3318.9183. 95% CI for the Mean: 4.81451<mean<4.95723. 95% CI for the Standard Deviation: 0.37912<SD<0.48083. 95% CI for the Variance: 0.14373<VAR<0.2312. FIG. 8B shows final tumor sizes for cisplatin with ImmunAG as adjuvant treatment. Sample Size, n: 138. Mean: 4.64362. Median: 4.615. Midrange: 4.745. RMS: 4.69632. Variance, s\hat{ }2: 0.49575. Standard Deviation, s: 0.70409. Mean Absolute Deviation: 0.57823. Range: 3.09. Coefficient of Variance: 15.16258%. Minimum: 3.2. 1st Quartile: 4.09. 2nd Quartile: 4.615. 3rd Quartile: 5.14. Maximum: 6.29. Sum: 640.82. Sum of Squares: 3043.644. 95% CI for the Mean: 4.5251<mean<4.76214. 95% CI for the Standard Deviation: 0.62968<SD<0.79861. 95% CI for the Variance: 0.3965<VAR<0.63778.

FIGS. 9A-9B are images of a cancer patient's PET scan before and after taking ImmunAG. FIG. 9A shows the patient's PET scan from Mar. 2, 2016, prior to taking ImmunAG. The bright white regions are metastasized cancer. FIG. 9B shows the patient's PET scan from Apr. 11, 2016, which shows 95% of the metastasized cancer had disappeared in 39 days after taking ImmunAG.

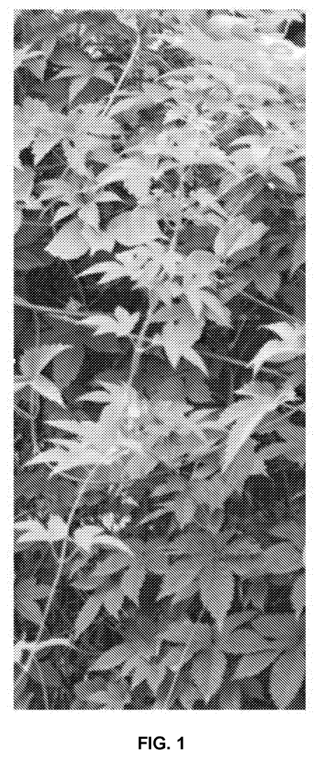

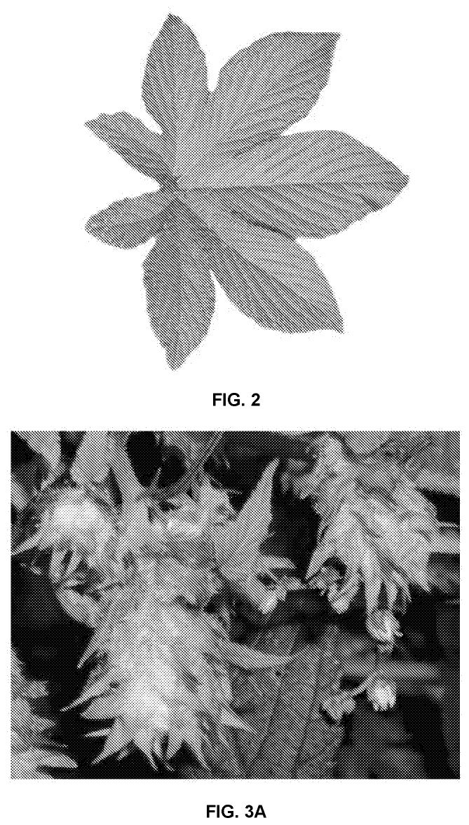

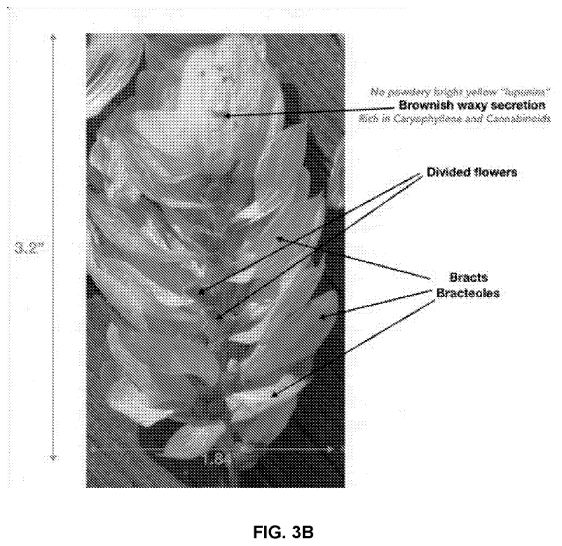

### DETAILED DESCRIPTION OF THE INVENTION

The present invention is directed to a Humulus plant bred to possess high concentrations of cannabinoids in its inflorescence, leaves, and other tissues. In accordance with this aspect of the present invention, the cannabinoid levels, in particular cannabidiol levels, in the tissues of the Humulus plant as described herein are significantly higher than the cannabinoid level in any corresponding Humulus plant found in nature.

Humulus is a genus of flowering plants in the family of Cannabaceae. The most common Humulus is Humulus lupulus, the “beer hops” flavor plant. The two other species of Humulus are Humulus scadens, also known as Humulus japonicus, native to Japan, and Humulus yunnanensis, native to the Himalayan mountain tracts bordering Nepal, India, and China. As described herein cannabinoids are a diverse class of compounds that are found primarily in Cannabis, another member of the Cannabaceae family. However, the presence of cannabinoids in Humulus lupulus or Humulus japonicus has not been reported. There is only one report, from the Central Food Technology Research Institute of India, located in Mysore India, which suggested that trace amounts of cannabinoids, including cannabidiol and cannabidivarin were found in a Humulus plant in the Hadamund Shola of Ooty India. As described in more detail in Example 1, a survey of Humulus yunnanensis plants from around India found low levels of cannabinoids (˜2.0 mg/g dry weight) in a small percentage (˜5%) of the H. yunnanensis population. Thus, applicant's Humulus plant, which was selectively bred to possess cannabinoid levels of >50 milligrams cannabinoids per gram dry weight in its inflorescence and >15 milligrams cannabinoids per gram dry weight in its leaves, is markedly different from any known naturally occurring Humulus plant.

The Humulus plant described herein can be a variant of any Humulus species, e.g., Humulus yunnanensis, Humulus japonicus, or Humulus lupulus, and any variety thereof (e.g., Humulus lupulus var. lupulus; H. lupulus var. cordifolius, H. lupulus var. lupuloides, H. lupulus var. neomexicanus, H. lupulus var. pubescens). In one embodiment, the Humulus plant as described herein is a variety of Humulus yunnanensis. An exemplary Humulus plant of the present invention is Humulus yunnanensis var kriya as described in Example 1 herein.

The term “cannabinoid” as understood by one of skill in the art, encompasses a class of diverse chemical compounds and derivatives thereof that exert activity on the cannabinoid receptors of cells, as well as posses a diverse range of other pharmacological properties. At least 113 different cannabinoids have been isolated from the Cannabis plant. Non-limiting examples of cannabinoids and cannabinoid products in Cannabis include cannabidiol (CBD), cannabigerol (CBG), cannabichromene (CBC), cannabielsoin (CBE), cannabicyclol (CBL), cannabinol (CBN), cannabicitran (CBT), tetrahydrocannabivarin (THCV), cannabivarin (CBV), cannabidivarin (CBDV), cannabichromevarin (CBCV), cannabigerovarin (CBGV), cannabigerol monomethyl ether (CBGM), tetrahydrocannabidiol or A⁹-tetrahydrocannabidiol (THC), iso-tetrahydrocannabinol (iso-THC), 11-hydroxy-Δ⁹-tetrahydrocannabinol (11-OH-Δ⁹-THC or 11-OH-THC), nabilone, and other cannabinoid analogs.

For the purpose of the present invention, reference to “cannabinoid”, “cannabinoids” and “cannabinoid level(s)” with respect to the Humulus plant described herein and extracts derived therefrom, explicitly excludes THC, iso-THC, 11-OH-Δ⁹-THC, 11-OH-THC, or any isomers, derivatives, or analogs thereof having psychoactive properties. In particular, the Humulus plant and extracts therefrom as described herein do not contain THC, iso-THC, 11-OH-Δ⁹-THC or 11-OH-THC, or any isomers, derivatives, or analogs thereof having psychoactive properties.

In one embodiment, the “cannabinoid level” of the Humulus plant and extracts thereof as described herein, is comprised one or more cannabinoids selected from the group consisting of cannabidiol (CBD), cannabigerol (CBG), cannabichromene (CBC), cannabielsoin (CBE) and cannabidivarin (CBDV). In one embodiment, the primary cannabinoid present in the Humulus plant as described herein is CBD.

Cannabidiol or CBD exerts diverse pharmacological activities via its interactions with a variety of cellular receptors. Cannabidiol acts primarily as an inverse agonist or partial agonist of the cannabinoid type-2 (CB₂) receptor. CBD is also an antagonist of the putative cannabinoid receptor, G protein-coupled receptor 55 (GPR55). CBD has also been shown to act as a 5-HT1A receptor agonist, and as an allosteric modulator at the Mu and Delta opioid receptor sites.

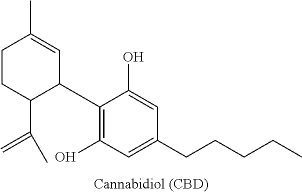 [SMILES: C=C(C)C1CCC(C)=CC1c1c(O)cc(CCCCC)cc1O]

While there are various isomers and stereoisomers of CBD, the primary, if not exclusive form of CBD found in the Humulus plant of the present invention, and extracts and compositions thereof is 2-(6-isopropenyl-3-methyl-2-cyclohexen-l-yl)-5-pentyl-1,3-benzenediol. This form of CBD is the only naturally occurring for of CBD, and consequently, the form of CBD having the highest level of bioactivity. CBD is known to exert an array of diverse actions, including, without limitation, suppressing inflammation and neuropathic pain, and reducing anxiety, stress, depression. CBD is also know to be an antiemetic, anticonvulsant, antipsychotic, anti-inflammatory, anti-tumoral, and an anti-bacterial agent

Cannabigerol has been found to act as a high affinity α2-adrenergic receptor agonist, a moderate affinity 5-HT1A receptor antagonist, and a low affinity CB₁ receptor antagonist. It also binds to the CB₂ receptor. Cannabigerol has been shown to relieve intraocular pressure, which may be of benefit in the treatment of glaucoma (Craig et al., “Intraocular Pressure, Ocular Toxicity and Neurotoxicity After Administration of Cannabinol or Cannabigerol” Exp. Eye Research 39 (3): 251-259 (1984, which is hereby incorporated by reference in its entirety). Cannabigerol has also been shown to reduce depression and be useful in the treatment of mood disorders (U.S. Pat. No. 8,481,085 to Musty and Deyo, which is hereby incorporated by reference in its entirety).

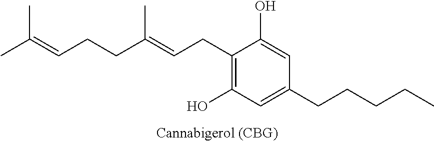 [SMILES: CCCCCc1cc(O)c(C/C=C(\C)CCC=C(C)C)c(O)c1]

Cannabichromene bears structural similarity to the other natural cannabinoids, including tetrahydrocannabinol, tetrahydrocannabivarin, cannabidiol, and cannabinol, among others. CBC is believed to possess anti-inflammatory and anti-viral properties as well as exert analgesic effects.

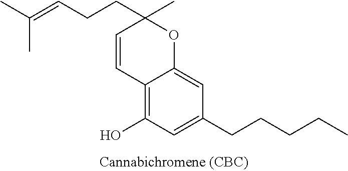 [SMILES: CCCCCc1cc(O)c2c(c1)OC(C)(CCC=C(C)C)C=C2]

Cannabidivarin (CBDV) is a non-psychoactive homolog of CBD, with the side-chain shortened by two methylene bridges (CH₂ units). CBDV has been found to reduce the number and severity of seizures in animal models of epilepsy (U.S. Pat. No. 9,125,859 to Whalley et al., which is hereby incorporated by reference in its entirety).

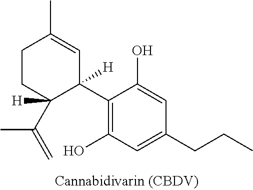 [SMILES: C=C(C)[C@@H]1CCC(C)=C[C@H]1c1c(O)cc(CCC)cc1O]

CBE is a metabolite of CBD (Yamamoto et al., “Cannabielsoin as a New Metabolite of Cannabidiol in Mammals,” Pharmacol. Biochem. Behav. 40(3): 541-6 (1991), which is hereby incorporated by reference in its entirety). Little is known about CBE's pharmacological activity.

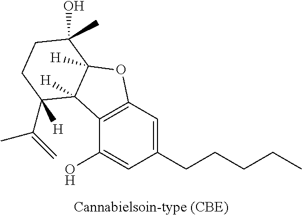

The Humulus plant as described herein contains high cannabinoid levels in its inflorescence and leaves. In one embodiment, the Humulus plant as described herein contains at least 50 milligrams of cannabinoids per gram of freeze-dried inflorescence. (mg/g dry weight). In another embodiment, the Humulus plant as described herein has a cannabinoid level of at least 55 mg/g, at least 60 mg/g, at least 65 mg/g, at least 70 mg/g, at least 75 mg/g, at least 80 mg/g, at least 85 mg/g, at least 90 mg/g, at least 95 mg/g, at least 100 mg/g, at least 105 mg/g, at least 110 mg/g, and least 115 mg/g, at least 120 mg/g, at least 125 mg/g, at least 130 mg/g, at least 135 mg/g, at least 140 mg/g, at least 145 mg/g dry weight of its inflorescence. In another embodiment, the inflorescence of the Humulus plant as described herein contains >145 mg cannabinoid per gram of freeze dried tissue. As noted above, the cannabinoid content is composed primarily of CBD, CBE, CBDV, CBG, and CBC, with CBD being the predominant cannabinoid.

In one embodiment, the Humulus plant as described herein further contains high cannabinoid levels in its leaves. In one embodiment, the Humulus plant as described herein contains at least 15 milligrams of cannabinoids per gram of freeze-dried leaf material (mg/g dry weight). In another embodiment, the Humulus plant as described herein has a cannabinoid level of at least 20 mg/g, at least 25 mg/g, at least 30 mg/g, at least 35 mg/g, at least 40 mg/g, at least 45 mg/g, at least 50 mg/g, and least 55 mg/g, at least 60 mg/g, at least 65 mg/g, at least 70 mg/g, at least 75 mg/g, at least 80 mg/g, at least 85 mg/g dry weight of its leaves. In another embodiment, the leaves of the Humulus plant as described herein contains >85 milligrams of cannabinoid per gram of freeze dried tissue.

The Humulus plant as described herein also contains high β-caryophyllene levels in its inflorescence and leaves. In one embodiment, the Humulus plant as described herein contains at least 20 milligrams of β-caryophyllene per gram of freeze-dried inflorescence (mg/g dry weight). In another embodiment, the Humulus plant as described herein has a β-caryophyllene level of at least 25 mg/g, at least 30 mg/g, at least 35 mg/g, at least 40 mg/g, at least 45 mg/g, at least 50 mg/g, at least 55 mg/g, and least 60 mg/g, at least 65 mg/g, at least 70 mg/g, at least 75 mg/g, at least 80 mg/g, at least 85 mg/g, at least 90 mg/g, at least 95 mg/g, at least 100 mg/g, at least 105 mg/g, at least 110 mg/g, at least 120 mg/g dry weight of its inflorescence. In another embodiment, the inflorescence of the Humulus plant as described herein contains >120 mg β-caryophyllene per gram of freeze dried tissue.

In one embodiment, the Humulus plant as described herein further contains high β-caryophyllene levels in its leaves. In one embodiment, the Humulus plant as described herein contains at least 5 milligrams of β-caryophyllene per gram of freeze-dried leaf material (mg/g dry weight). In another embodiment, the Humulus plant as described herein has a β-caryophyllene level of at least 10 mg/g, at least 15 mg/g, at least 20 mg/g, at least 25 mg/g, at least 30 mg/g, at least 35 mg/g, at least 40 mg/g dry weight of its leaf material. In another embodiment, the leaves of the Humulus plant as described herein contains >40 milligrams of β-caryophyllene per gram of freeze dried tissue.

The present invention also encompasses isolated Humulus plant components, Humulus plant extracts, and compositions comprising these Humulus components and extracts. Accordingly, another aspect of the present invention is directed to an extract of the Humulus plant described herein. In accordance with this aspect, the extract is an extract derived from one or more tissues of the Humulus plant, for example, and without limitation, from one or more of the inflorescence, the leaves, the stem, the bark, the roots, the shoot tips, or any combination thereof. In one embodiment, the extract is an extract derived from the inflorescence of the Humulus plant described herein. While such extracts are preferably obtained from the inflorescence of the plant during the late vegetative or early flowering stage, extracts as described herein may be obtained from the inflorescence and any other plant tissue during any period of its growth cycle.

Methods of extracting cannabinoids and other compounds of interest from plant material are well known in the art and applicable to extracting cannabinoids and other compounds of interest from the Humulus plant of the present invention, see e.g., WO2016153347 to Martinez et al., U.S. Pat. No. 8,895,078 to Mueller, U.S. Pat. No. 7,344,736 to Whittleband, and U.S. Pat. No. 7,700,368 to Flockhart, which are hereby incorporated by reference in their entirety. These methods of extracting cannabinoids and other compounds from the Humulus plant material can be utilized to obtain the Humulus plant extracts described herein.

In one embodiment, the Humulus plant extract as described herein comprises cannabidiol (CBD). As noted above, CBD exerts an array of diverse actions, including, without limitation, suppressing inflammation and neuropathic pain, and reducing anxiety, stress, depression. CBD is also know to be an antiemetic, anticonvulsant, antipsychotic, anti-inflammatory, anti-tumoral, and an anti-bacterial agent.

In another embodiment, the Humulus plant extract as described herein comprises β-caryophyllene. β-caryophyllene is a natural bicyclic sesquiterpene that is found in the essential oils of many plants. It is known to be a selective agonist of the cannabinoid receptor type-2 and to exert biological activity such as anti-inflammatory, antibiotic, antioxidant, anticarcinogenic, and local anaesthetic activity.

In another embodiment, the Humulus plant extract as described herein comprises humulene. Humulene, also known as α-humulene or α-caryophyllene, is a naturally occurring monocyclic sesquiterpene. Humulene is found in in many aromatic plants, including Humulus lupulus, and is known for its “hoppy” aroma. However, humulene is also known to have biological activity, e.g., anti-inflammatory anti-bacterial, and anti-cancer activity

In another embodiment, the Humulus plant extract as described herein comprises a combination of CBD, β-caryophyllene, and/or humulene. In another embodiment, the Humulus plant extract comprises one or more of CBD, β-caryophyllene, and/or humulene in combination with one or more other compounds of the Humulus plant described herein including 6-prenylnaringenin, 8-prenylnaringenin, adhumulone, adlupulone, cannabichromene, cannabidivarin, cannabigerol, farnesene, humulone, isoxanthohumol, lupulone, xanthohumol, or any combination thereof. In one embodiment, the Humulus plant extract contains humulene, CBD, β-caryophyllene, 6-prenylnaringenin, 8-prenylnaringenin, adhumulone, adlupulone, cannabichromene, cannabidivarin, cannabigerol, farnesene, humulone, isoxanthohumol, lupulone, and xanthohumol. Example 2 herein provides an exemplary compositional make up of a Humulus plant extract of the present invention.

Another embodiment of the present invention is directed to a composition comprising CBD, β-caryophyllene, and/or humulene, and one or more of the following compounds: 6-prenylnaringenin, 8-prenylnaringenin, adhumulone, adlupulone, cannabichromene, cannabidivarin, cannabigerol, farnesene, humulone, isoxanthohumol, lupulone, and xanthohumol. In another embodiment, the composition of the present invention comprises humulene, CBD, β-caryophyllene, 6-prenylnaringenin, 8-prenylnaringenin, adhumulone, adlupulone, cannabichromene, cannabidivarin, cannabigerol, farnesene, humulone, isoxanthohumol, lupulone, and xanthohumol. In one embodiment, the composition comprises or consists essentially of about 9% humulene, about 12% CBD, about 14% β-caryophyllene, about 6% 6-prenylnaringenin, about 4% 8-prenylnaringenin, about 5% adhumulone, about 6% adlupulone, about 5% cannabichromene, about 2% cannabidivarin, about 2% cannabigerol, about 5% farnesene, about 9% humulone, about 6% isoxanthohumol, about 8% lupulone, and about 7% xanthohumol. This composition, like all other compositions of the present invention, does not contain tetrahydrocannabinolic acid (THCA), tetrahydrocannabinol (THC), or any isomers or derivatives thereof having psychoactive properties.

In another embodiment of the present invention, the humulene, β-caryophyllene, and cannabidiol components of the Humulus plant extract are isolated or purified to provide a substantially pure preparation comprising these three components. The precise content and purity of any particular Humulus plant material and/or extract may be qualitatively and quantitatively determined using analytical techniques well known to those skilled in the art, such as thin-layer chromatography (TLC) or high performance liquid chromatography (HPLC).

As referred to herein, a “substantially pure” preparation is defined herein as a preparation of cannabidiol, humulene, and β-caryophyllene having a chromatographic purity of greater than 90%, preferably greater than 95%, preferably greater than 96%, more preferably greater than 97%, more preferably greater than 98%, more preferably greater than 99% and most preferably greater than 99.5%, as determined by its HPLC profile.

In accordance with this aspect of the present invention, the purified Humulus plant extract comprises a cannabidiol content of 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, or >90%; a (3-caryophyllene content of 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, or >90%; and a humulene content of 0.5%, 1%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, 20.0%, 21.0%, 22.0%, 23.0%, 24.0%, 25.0%, or >25% humulene.

In accordance with this aspect of the present invention, the purified Humulus extract comprises 5%-90% cannabidiol, 5%-90% β-caryophyllene, and 1%-10% humulene. In another embodiment, the purified Humulus extract comprises 20%-70% cannabidiol, 30%-80% β-caryophyllene, and 1%-8% humulene. In another embodiment, the purified Humulus extract comprises 25%-55% cannabidiol, 45%-75% β-caryophyllene, and 1%-5% humulene. In another embodiment, the purified Humulus extract comprises 30%-45% cannabidiol, 50%-70% β-caryophyllene, and 1%-5% humulene. In another embodiment, the purified humulene extract comprises 38.5% cannabidiol, 60% β-caryophyllene, and 1.5% humulene. In another embodiment, the purified humulene extract consists essentially of 38.5% cannabidiol, 60% β-caryophyllene, and 1.5% humulene.

Yet another aspect of the present invention is directed a composition comprising humulene, β-caryophyllene, and cannabidiol as described above. This composition does not contain THCA, THC, or any isomers or derivatives thereof having psychoactive properties.

Another aspect of the present invention is directed to pharmaceutical, nutraceutical, and cosmetic compositions comprising any of the Humulus plant extracts as described above. Formulations of these compositions and methods of their use are described infra.

The compositions as described herein can be formulated for administration to a human and/or animal subject that is in need thereof. Such formulations encompass pharmaceutical formulations, nutraceutical formulations, and cosmetic formulations. As is understood by those skilled in the art the term “pharmaceutical formulation” refers to a composition that is designed for administration to a human or animal for therapeutic purposes, typically for the treatment and/or prevention of a condition or symptoms of that condition. A “nutraceutical formulation” is a composition that is designed for administration to a human and/or animal subject for nutritional, dietary, or health purposes. The term “cosmetic formulation” refers to a composition applied externally, e.g. to the skin, nails, or hair of the human or animal body, for the purpose of cleaning, conditioning, or protecting the integrity and appearance of the bodily surface.

The various formulations of the present invention may comprise 10% (wt), 20%, 25%, 35%, 30%, 40%, 45%, 50%, 55%, 60%, 65%, 70%, 75%, 80%, 85%, 90%, or >90% by wt of the Humulus plant extract as described herein.

Generally, the Humulus plant extracts and compositions of the present invention will be administered as a formulation in association with one or more physiologically acceptable excipients. The term “excipient” as used herein refers to any ingredient other than the Humulus plant extract or composition thereof. In particular, excipients are generally, but not always, inert substances added to a formulation to further facilitate administration of the active ingredient(s), e.g., CBD, β-caryophyllene, and humulene. Excipients are well known in the art, and are used in a variety of formulations. Common excipients include diluents, such as lactose, dextrin, glucose, sucrose, sorbitol, silicates, calcium and magnesium salts, sodium or potassium chloride; binders, compression aids, and granulating agents such as natural or synthetic polymers (e.g., starches, sugars, sugar alcohols, and cellulose derivatives); disintegrants such as starch, cellulose derivatives, and alginates; glidants such as colloidal anhydrous silicon and other silica compounds; lubricants such as stearic acid and its salts; tablet coatings and films such as sugars and polymers; colouring agents and dyes. The choice of excipient will, to a large extent, depend on factors such as the particular mode of administration, the effect of the excipient on solubility and stability, and the nature of the dosage form.

In one embodiment, the formulation comprises the Humulus plant extract in conjunction with a physiologically acceptable carrier or diluent. Suitable pharmaceutically acceptable carriers include oil, starch, glucose, lactose, sucrose, gelatin, malt, rice, flour, chalk, silica gel, sodium stearate, vegetable stearate, glycerol monostearate, talc, sodium chloride, dried skim milk, hydration salts, glycerol, propylene glycol and the like. In one embodiment, the carrier is oil. In one embodiment, the oil carrier is coconut oil.

Formulations suitable for the delivery of the Humulus extracts and compositions as described herein, and methods for their preparation will be readily apparent to those skilled in the art. Such compositions and methods for their preparation may be found, for example, in REMINGTON'S PHARMACEUTICAL SCIENCES, 22nd ed. (Pharmaceutical Press, 2013), which is hereby incorporated by reference in its entirety. Formulations will be optimized for a particular route of administration. Suitable routes of administration include, without limitation oral administration; topical administration; transdermal administration; ocular administration; transmucosal administration, especially transnasal, bronchial, pulmonary, buccal, sublingual, rectal and vaginal; parenteral administration, including intramuscular, subcutaneous and intramedullary injections as well as intrathecal, direct intraventricular, intravenous and intraperitoneal injections.

In one embodiment, formulations described herein are suitable for transdermal administration. The transdermally administrable formulations can be adapted for administration in and/or around the abdomen, back, chest, legs, arms, hands, feet, joints, scalp, behind the ear, neck, jaw, or other suitable skin surface and may include formulations in which the Humulus extracts and compositions comprising the same are administered in patches, ointments, creams, suspensions, lotions, pastes, gels, sprays, foams or oils.

In one embodiment, formulations described herein are suitable for topical administration. Topically administrable formulations can be adapted for administration in and/or around the abdomen, back, chest, legs, arms, hands, feet, joints, scalp, behind the ears, neck, jaw or other suitable skin surface and may include formulations in which the Humulus extracts and compositions comprising the same are administered in patches, ointments, creams, suspensions, lotions, pastes, gels, sprays, foams or oils. In one embodiment, the formulation is an oil-in-water emulsion. In another embodiment, the formulation is an oil-in-oil blend. In another embodiment, the formulation is a water-in-oil emulsion.

In one embodiment, topical formulations described herein are intended for cosmetic applications. Such cosmetic formulations may include shampoos, lotions, creams, moisturizers, gels, sun screens, makeup, cleansers, soaps, foams, oils, pastes, sprays, and patches.

In another embodiment, the formulations described herein are suitable for oral administration. Compositions described herein that are orally administrable include formulations in which the Humulus extracts and compositions thereof are administered in tablets, capsules, suspensions, syrups or liquids, powder, granules, or oil. In another embodiment, the formulations may be formulated as extended release, sustained release, or long acting tablet or capsule. Methods of making sustained release tablets are known in the art; see e.g., U.S. Patent Publication No. 2006/0051416 to Rastogi, and U.S. Patent Publication No. 2007/0065512 to Dedhiya, which are hereby incorporated by reference in their entirety. Gradual-release tablets are also known in the art; examples of such tablets are set forth in U.S. Pat. No. 3,456,049 to Hotko, which is hereby incorporated by reference in its entirety. A slow- or sustained-release form may delay disintegration or absorption of the composition or one or more components thereof.

Compositions suitable for oral administration, e.g., tablets, capsules, etc., comprise a concentration of cannabidiol, β-caryophyllene, and humulene independently selected from 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, or >100 mg. In one embodiment, the oral formulation comprises 0.5-3.0 mg humulene, 1.0-5.0 mg β-caryophyllene, and 7.5-100 mg cannabidiol. In one embodiment, the oral formulation comprises 1.5 mg humulene, 3 mg β-caryophyllene, and 7.5 mg cannabidiol. In another embodiment, the oral formulation comprises 1.5 mg humulene, 3 mg β-caryophyllene, and 40 mg cannabidiol. In another embodiment, the oral formulation comprises 1.5 mg humulene, 3 mg β-caryophyllene, and 100 mg cannabidiol.

In one embodiment, the oral formulation is a capsule. The capsule contains a plant extract as described herein in carrier oil. The carrier can be any food grade oil. In one embodiment, the carrier oil is coconut oil. In accordance with this embodiment, the capsules contain 7.5 mg cannabidiol (12.5 mg/capsule total cannabinoids), 40 mg cannabidiol (67 mg/capsule total cannabinoids), or 100 mg cannabidiol (167 mg/capsule total cannabinoids). The capsules may further comprise 1.5 mg humulene and/or 3 mg β-caryophyllene. In one embodiment, the capsule comprises cannabidiol (7.5 mg, 40 mg, or 100 mg) in combination with 1.5 mg humulene and 3 mg β-caryophyllene.

In another embodiment, the oral formulation is a tablet. In one embodiment, the tablet is a time release tablet formulation. In accordance with this embodiment, the tablet contain 7.5 mg cannabidiol (12.5 mg/tablet total cannabinoids), 40 mg cannabidiol (67 mg/tablet total cannabinoids), or 100 mg cannabidiol (167 mg/tablet total cannabinoids). The tablet may further comprise 1.5 mg humulene and/or 3 mg β-caryophyllene. In one embodiment, the tablet comprises cannabidiol (7.5 mg, 40 mg, or 100 mg) in combination with 1.5 mg humulene and 3 mg β-caryophyllene.

In one embodiment, formulations described herein are suitable for buccal administration. Formulations described herein that are buccally administrable may include formulations in which the Humulus extracts and compositions are administered in lozenges, sprays, gels, pastes, dissolvable tablets or dissolvable strips. In another embodiment, formulations described herein are suitable for sublingual administration. Sublingual formulations described herein may include formulations in which the Humulus extracts and compositions are administered in lozenges, sprays, gels, pastes, dissolvable tablets or dissolvable strips.

In one embodiment, formulations described herein are suitable for injectable administration. Formulations described herein that are suitable for injectable administrable may include formulations in which the Humulus extracts and compositions are administered as an intravenous, intrathecal, subcutaneous or depot injection.

In one embodiment, formulations described herein are suitable for bronchial and pulmonary administration. Formulations described herein that are suitable for pulmonary administration may include formulations in which the Humulus extracts and compositions are administered as an aerosol, pressurized atomizers, inhalers of dry powder, or dissolved in volatile liquids.

In one embodiment, formulations described herein are suitable for rectal administration. Formulations described herein that are rectally administrable may include formulations in which the Humulus extracts and compositions are placed in suppositories, ointments, creams, suspensions, solutions, lotions, pastes, gels, sprays, foams or oils.

In one embodiment, formulations described herein are suitable for vaginal administration. Formulations described herein that are vaginally administrable may include formulations in which the Humulus extracts and compositions are placed in suppositories, ointments, creams, suspensions, solutions, lotions, pastes, gels, sprays, foams or oils.

In one embodiment, formulations described herein are suitable for ocular administration. Formulations described herein that are ocularly administrable may include formulations in which the Humulus extracts and compositions are placed in ointments, suspensions, solutions, gels or sprays.

In one embodiment, formulations described herein are suitable for nasal administration. Formulations described herein that are nasally administrable may include formulations in which the Humulus extracts and compositions are placed in ointments, suspensions, solutions, lotions, pastes, gels, sprays or mists.

Formulations suitable for administration to a subject in need thereof, e.g., via transdermal, topical, mucosal, ocular, nasal formulations, comprise a concentration of cannabidiol, β-caryophyllene, and humulene independently selected from 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 3 lmg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, or >100 mg.

A further aspect of the present invention concerns methods of therapeutic and/or non-therapeutic treatment of a human or animal subject in need thereof, said method comprising administering to said human or animal subject in need thereof an extract of the Humulus plant or composition comprising said extract as described herein.

In one embodiment, the Humulus plant extracts and compositions comprising the same are utilized by a subject as a dietary and/or health supplement for the purpose of boosting or enhancing their immune system function, cardiovascular health, renal system health, nervous system health, endocrine system health, digestive health, vascular health, etc. In one embodiment, the Humulus plant extracts and compositions comprising the same are utilized to promote homeostasis of the body as a whole.

In another embodiment, the present invention is directed to a method of modulating G-protein coupled receptor activity in a subject. This method involves selecting a subject in need of G-protein coupled receptor activity modulation, and administering to said selected subject, an extract of the Humulus plant or composition comprising said extract as described herein, in amount effective to modulate G-protein coupled receptor (GPCR) activity in the subject.

CBD is known to modulate GPCR activity directly as well as indirectly via modulation of ligands and enzymes of the GPCR. Thus, administration of the Humulus plant extract or composition comprising the same as described herein can be administered to inhibit or block the activity of fatty acid amide hydrolase, to antagonize the anandamide reuptake inhibitor, antagonize GPR55, antagonize TRPM8, antagonize adenosine uptake. The Humulus plant extract or composition comprising the same can also be administered to modulate the activity of M opioid receptor (inverse agonist), α1 and α1β glycerin receptors (agonist), CB2 (agonist or inverse agonist), TRPA1 (agonist), TRPV1 (agonist), TRPV2 (agonist), PPAR-gamma (agonist), 5HT1A (agonist), T-type Ca⁺² channel (inhibitor), 5-lipoxygenase (inhibitor), 15-lipoxygenase (inhibitor), phospholipase A2, CXCL8, CCL2, CCL3, CCL4, CCLS, CCL11, CXCL10, angiotensin II receptor (agonist), somatostatin receptors 1-5 (inverse agonist), galanin receptors (partial agonist), cysteinyl leukotriene receptor 1 and 2 (agonist), leukotriene B4 receptors 1 and 2 (partial antagonist), relaxin/insulin-like family peptide receptors 1, 2, 3, 4 (agonist), KiSS1-derived peptide receptor (GPR54) (inverse agonist), melanin-concentrating hormone receptor 1 (inverse agonist), urotensin II receptor (agonist), ACTH receptor (reuptake inhibitor), Lysophosphatidic acid receptor 1, 2, 3 (inhibitor), Sphingosine 1-phosphate receptor 1, 2, 3, 4, 5 (reuptake inhibitor), Melanocortin/ACTH receptor 1,2,3,4,5 (partial agonist), adrenergic receptor (agonist), and histamine H1 receptor (HRH1, HRH3, HRH4) (inverse agonist).

In another embodiment, the present invention is directed to a method of modulating endocannabinoid system activity in a subject. This method involves selecting a subject in need of endocannabinoid system modulation, and administering to said selected subject, an extract of the Humulus plant or composition comprising said extract as described herein, in amount effective to modulate endocannabinoid system activity in the subject.

In accordance with this aspect of the present invention, administering an extract of the Humulus plant or composition comprising said extract as described herein increases endocannabinoid system activity in the subject. In one embodiment, an extract of the Humulus plant or composition comprising said extract as described herein modulates the activity of the cannabinoid type-2 receptor (CB₂ receptor). In one embodiment, an extract of the Humulus plant or composition comprising said extract as described herein increases the activity of the CB₂ receptor.

In one embodiment, the subject in need of endocannabinoid system modulation is a subject suffering from stress, anxiety, a sleep disorder, a mood disorder, epilepsy or other seizure disorder, schizophrenia, autism, and/or depression. Administering an extract of the Humulus plant or composition comprising said extract as described herein reduces or alleviates one or more symptoms of the aforementioned conditions, reduces the severity of the condition, and/or alleviates the condition in its entirety.

In one embodiment, administering an extract of the Humulus plant or composition comprising said extract as described herein to a patient having epilepsy or other seizure disorder reduces the frequency of the epileptic events and, in some instances, stops the epileptic events altogether.

In another embodiment, the subject in need of endocannabinoid system modulation is a subject suffering from chronic pain, migraines, or neuropathy. Administering an extract of the Humulus plant or composition comprising said extract as described herein reduces or alleviates one or more symptoms of the chronic pain, migraines, or neuropathy, reduces the severity of the condition, and/or alleviates the condition in its entirety.

In another embodiment, the subject in need of endocannabinoid system modulation is a subject suffering from lock jaw or temporomandibular joint disease, and administering an extract of the Humulus plant or composition comprising said extract as described herein reduces or alleviates one or more symptoms of the condition, reduces the severity of the condition, and/or alleviates the condition in its entirety.

In another embodiment, the subject in need of endocannabinoid system modulation is a subject suffering from addiction or anorexia. Administering an extract of the Humulus plant or composition comprising said extract as described herein reduces or alleviates one or more symptoms of the aforementioned conditions, reduces the severity of the condition, and/or alleviates the condition in its entirety.

In another embodiment, the subject in need of endocannabinoid system modulation is a subject suffering from an inflammatory condition. Inflammatory conditions suitable for treatment with an extract or composition of the present invention include chronic and acute inflammatory conditions. Exemplary inflammatory conditions to be treated include, without limitation, irritable bowel syndrome, arthritis (including rheumatoid arthritis and psoriatic arthritis), asthma, Crohn's disease, colitis, piles (hemorrhoids), ischemia/reperfusion injury. Other inflammatory conditions that can be treated with the extracts and compositions described herein include atherosclerosis, psoriasis, multiple sclerosis, lupus, type I diabetes, primary biliary cirrhosis, inflammatory bowel disease, tuberculosis, skin wounds and infections, tissue abscesses, folliculitis, osteomyelitis, pneumonia, scalded skin syndrome, septicemia, septic arthritis, myocarditis, endocarditis, toxic shock syndrome, allergic contact dermatitis, acute hypersensitivity, acute neurological inflammatory injury, conjunctivitis, iritis, uveitis, central retinitis, external otitis, acute suppurative otitis media, mastoiditis, labyrinthitis, chronic rhinitis, acute rhinitis, sinusitis, pharyngitis, tonsillitis, contact dermatitis, dermonecrosis, diabetic polyneuritis, polymyositis, myositis ossificans, periarthritis, and osteitis deformans. Administering an extract of the Humulus plant or composition comprising said extract as described herein reduces or alleviates one or more symptoms of the aforementioned inflammatory conditions, reduces the severity of the condition, and/or alleviates the condition in its entirety.

In one embodiment, the subject in need of endocannabinoid system modulation is a subject suffering from asthma. Administering an extract of the Humulus plant or composition comprising said extract as described herein to a patient having asthma reverses the symptoms of asthma and improves breathing.

In another embodiment, the subject in need of endocannabinoid system modulation is a subject suffering from diabetes, and administering an extract of the Humulus plant or composition comprising said extract as described herein reduces or alleviates one or more symptoms of diabetes, reduces the severity of the condition, and/or alleviates the condition in its entirety.

In another embodiment, the subject in need of endocannabinoid system modulation is a subject suffering from neurodegenerative disease, such as Alzheimer's disease, Parkinson's disease, prion disease, or Huntington's disease. Administering an extract of the Humulus plant or composition comprising said extract as described herein reduces or alleviates one or more symptoms of the aforementioned neurodegenerative disease, reduces the severity of the disease, and/or alleviates the disease in its entirety. In one embodiment, administering an extract of the Humulus plant or composition comprising said extract as described herein to patients having a neurodegenerative disease, e.g., Alzheimer's disease, reverses degeneration and improves cognitive test scores.

In another embodiment, the subject in need of endocannabinoid system modulation is a subject suffering from cancer. Cancers that can be treated with the extracts and compositions comprising the same as described herein include, without limitation, acute lymphoblastic leukemia, acute myeloid leukemia, adrenocortical carcinoma, AIDS related sarcoma, AIDS-related lymphoma, anal cancer, appendix cancer, astrocytoma (childhood cerebellar or cerebral), basal-cell carcinoma, bile duct cancer, bladder cancer, bone tumor, osteosarcoma/malignant fibrous histiocytoma, brainstem glioma, brain cancer, brain tumor, ependymoma, medulloblastoma, supratentorial primitive neuroectodermal tumors, visual pathway and hypothalamic glioma, breast cancer, bronchial adenomas/carcinoids, Burkitt's lymphoma, carcinoid tumor (childhood), carcinoid tumor (gastrointestinal), carcinoma of unknown primary, central nervous system lymphoma, cervical cancer, childhood cancers, chondrosarcoma, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, epitheliod hemangioendothelioma (EHE), esophageal cancer, Ewing's sarcoma, extracranial germ cell tumor, extragonadal germ cell tumor, extrahepatic bile duct cancer, intraocular melanoma, retinoblastoma, gallbladder cancer, gastric (stomach) cancer, gastrointestinal carcinoid tumor, gastrointestinal stromal tumor (GIST), germ cell tumor, gestational trophoblastic tumor, glioma of the brain stem, gastric carcinoid, hairy cell leukemia, head and neck cancer, heart cancer, hepatocellular (liver) cancer, hodgkin lymphoma, hypopharyngeal cancer, intraocular melanoma, islet cell carcinoma (endocrine pancreas), Kaposi sarcoma, kidney cancer (renal cell cancer), laryngeal cancer, lip and oral cavity cancer, liposarcoma, non-small cell lung cancer, small cell lung cancer, lymphoma, AIDS-related lymphoma, cutaneous T-Cell lymphoma, male breast cancer, melanoma, merkel cell cancer, mesothelioma, metastatic squamous neck cancer with occult primary, mouth cancer, multiple endocrine neoplasia syndrome, multiple myeloma/plasma cell neoplasm, Mycosis fungoides, myelodysplastic syndromes, myelodysplastic/myeloproliferative diseases, multiple myeloma, chronic myeloproliferative disorder, myxoma, nasal cavity and paranasal sinus cancer, nasopharyngeal carcinoma, neuroblastoma, oligodendroglioma, oral cancer, oropharyngeal cancer, ovarian cancer, pancreatic cancer, parathyroid cancer, penile cancer, pharyngeal cancer, pheochromocytoma, pineal astrocytoma, pineal germinoma, pineoblastoma and supratentorial primitive neuroectodermal tumors, pituitary adenoma, plasma cell neoplasia/multiple myeloma, pleuropulmonary blastoma, primary central nervous system lymphoma, prostate cancer, rectal cancer, retinoblastoma, rhabdomyosarcoma, salivary gland cancer, soft tissue sarcoma, uterine sarcome, Sézary syndrome, skin cancer (non-melanoma), small intestine cancer, squamous cell carcinoma, squamous neck cancer with occult primary, stomach cancer, supratentorial primitive neuroectodermal tumor (childhood), T-Cell lymphoma (cutaneous), testicular cancer, throat cancer, thymoma and thymic carcinoma, thyroid cancer, transitional cell cancer of the renal pelvis and ureter, trophoblastic tumor, urethral cancer, uterine cancer (endometrial), uterine sarcoma, vaginal cancer, vulvar cancer, Waldenström macroglobulinemia, and Wilms tumor (kidney cancer), combinations of said cancers, and metastatic lesions of said cancers. Administering an extract of the Humulus plant or composition comprising said extract as described herein reduces or alleviates one or more symptoms of the aforementioned cancers, reduces the severity of the cancer, reduces or prevents metastasis of the cancer, and/or alleviates the cancer in its entirety. In one embodiment, administering an extract of the Humulus plant or composition comprising said extract as described herein minimizes the side effects of chemotherapy.

In another embodiment, the subject in need of endocannabinoid system modulation is a subject suffering from a skin condition such as, without limitation, acne, psoriasis, skin allergy, or pruritus. Administering an extract of the Humulus plant or composition comprising said extract as described herein reduces or alleviates one or more symptoms of the aforementioned skin conditions, reduces the severity of a condition, and/or alleviates the condition in its entirety.

In another embodiment, the subject in need of endocannabinoid system modulation is a subject suffering from obesity. Administering an extract of the Humulus plant or composition comprising said extract as described herein facilitates weight loss and/or prevents further weight gain by the subject.

In another embodiment, the subject in need of endocannabinoid system modulation is a subject suffering from a movement disorder, e.g., dyskinesia. Administering an extract of the Humulus plant or composition comprising said extract as described herein reduces or alleviates one or more symptoms of the movement disorder, reduces the severity of the disorder, and/or alleviates the disorder in its entirety.

In another embodiment, the subject in need of endocannabinoid system modulation is a subject suffering from a brain or spinal cord injury. Administering an extract of the Humulus plant or composition comprising said extract as described herein reduces or alleviates one or more symptoms of the aforementioned conditions, facilitates healing and reduces the severity of a condition, and/or alleviates the condition in its entirety.

In another embodiment, the subject in need of endocannabinoid system modulation is a subject suffering from glaucoma, and administering an extract of the Humulus plant or composition comprising said extract as described herein reduces or alleviates one or more symptoms of glaucoma, reduces the severity of the glaucoma, and/or alleviates the condition in its entirety. In one embodiment, administering an extract of the Humulus plant or composition comprising said extract as described herein significantly reduces internal eye pressure.

In another embodiment, the subject in need of endocannabinoid system modulation is a subject suffering from chemotherapy induced toxicity. Administering an extract of the Humulus plant or composition comprising said extract as described herein reduces or alleviates one or more symptoms of the aforementioned toxicity, reduces the severity of the toxicity, and/or alleviates the toxicity in its entirety.

In another embodiment, the subject in need of endocannabinoid system modulation is a subject suffering from cardiovascular disease and in particular, a cardiovascular arrhythmia. Administering an extract of the Humulus plant or composition comprising said extract as described herein reduces or alleviates one or more symptoms of the arrhythmia, reduces the severity or frequency of the arrhythmic condition, and/or alleviates the condition in its entirety.

In another embodiment, the subject in need of endocannabinoid system modulation is a subject suffering from renal degeneration and/or renal disease such as nephritis. Administering an extract of the Humulus plant or composition comprising said extract as described herein reduces or alleviates one or more symptoms of the renal degeneration and/or renal disease, reduces the severity of the condition, and/or alleviates the condition in its entirety. In one embodiment, administering an extract of the Humulus plant or composition comprising said extract as described herein to a patient having nephritis improves renal filtration.

In another embodiment, the subject in need of endocannabinoid system modulation is a subject suffering from lyme disease. Administering an extract of the Humulus plant or composition comprising said extract as described herein reduces or alleviates one or more symptoms of the lyme disease, reduces the severity of the disease, and/or alleviates the disease in its entirety.

Effective doses of the Humulus plant extracts and compositions containing the same of the present invention for the treatment of the above described conditions will vary depending upon many different factors, including mode and frequency of administration, the concentrations of active agents in the composition, target site, physiological state of the patient (including sex, height, and weight), whether the patient is human or an animal, other medications administered, and whether treatment is prophylactic or therapeutic. Usually, the subject is a human, but in some diseases, the subject can be a nonhuman mammal. Non-human mammals amenable to treatment in accordance with the methods of the present invention include primates, dogs, cats, rodents (e.g., mouse, rat, guinea pig), horses, deer, cervids, cattle and cows, sheep, and pigs. Treatment dosages need to be titrated to optimize safety and efficacy. General guidance can be found, for example, in REMINGTON'S PHARMACEUTICAL SCIENCES (Mack Publishing Company 1990), which is hereby incorporated by reference in its entirety.

Compositions of the present invention may be administered in a single dose, or in accordance with a multi-dosing protocol. For example, in one embodiment of the present invention, relatively few doses of a composition are administered, such as one or two doses. In another embodiment of the present invention, the therapeutic composition is administered more frequently, e.g., hourly, daily, weekly, monthly, etc. until the desired therapeutic benefit is achieved. However, the different dosages, timing of dosages, and relative amounts of the composition can and should be selected and adjusted by one of ordinary skill in the art based on the subject and condition being treated.

### EXAMPLES

### Example 1—Generation and Characterization of Humulus yunnanensis var. kriya

A new and distinct Humulus plant described herein and designated as ‘kriya’ was produced from a cross hybridization of feral H. yunnanensis variants collected from the Pekong area within the Arunachal Pradesh region of India. Various H. yunnanensis samples were collected for analysis from various regions of India, including the groves in Puging, Singing, and Pekong, as well as in Mouling National Park, Kaying, and Lipo. H. yunnanensis male and female saplings with roots were collected, along with male and female flowers. All collected samples were tested for the presence of cannabinoids using standard methods known in the art (see Korte. F. and Sieper. H., J. Chromatoqr. 13:90 (1964), which is hereby incorporated by reference in its entirety). Of 1,174 H. yunnanensis plant samples collected and tested, only 61 (5.2%) of the samples contained detectable levels of cannabinoids. See Table 1 below. Moreover, average cannabinoid level in the inflorescence of the H. yunnanensis plants containing cannabinoids was 2.1 mg/g¹. ¹The cannabinoid level in plant tissue as described throughout this application is provided as milligrams of cannabinoid per gram of freeze dried plant material.

TABLE 1
Comparison of Cannabinoid Content in Samples of H. yunnanensis Collected From India and Bhutan

| | Ooty¹ | | | | | | | | | |
|---|---|---|---|---|---|---|---|---|---|---|
| | | Samples with | Puging² | | Singing³ | | Pekong⁴ | | Mouling⁵ | |
| | Samples | Cannabinoids | Samples | Samples | Samples | Samples | Samples | Samples | Samples | Samples |
| | collected | (“CB”) | collected | w/CB | collected | w/CB | collected | w/CB | collected | w/CB |
| Flowers | 19 | 0 | 23 | 0 | 41 | 0 | 69 | 14 | 31 | 4 |
| Shoot tips | 11 | 0 | 18 | 0 | 32 | 0 | 36 | 8 | 23 | 2 |
| Leaves | 23 | 0 | 36 | 0 | 67 | 0 | 103 | 13 | 47 | 2 |
| Stem | 15 | 0 | 11 | 0 | 24 | 0 | 48 | 0 | 12 | 0 |
| Bark | 9 | 0 | 8 | 0 | 20 | 0 | 29 | 0 | 8 | 0 |
| Roots | 6 | 0 | 3 | 0 | 8 | 0 | 15 | 0 | 0 | 0 |
| Total | 83 | | 99 | | 192 | | 300 | | 121 | |
| Collected | | | | | | | | | | |
| Samples | | 0 | | 0 | | 0 | | 35 | | 8 |
| w/CB | | | | | | | | | | |

| | Kaying⁶ | | Lipo⁷ | | Bomdeling⁸ | | Nanda Devi⁹ | | |
|---|---|---|---|---|---|---|---|---|---|
| | Samples | Samples | Samples | Samples | Samples | Samples | Samples | Samples | % with |
| | collected | w/CB | collected | w/CB | collected | w/CB | collected | w/CB | CB |
| Flowers | 29 | 0 | 19 | 4 | 18 | 0 | 11 | 2 | |
| Shoot tips | 21 | 0 | 14 | 2 | 14 | 0 | 15 | 2 | |
| Leaves | 35 | 0 | 35 | 6 | 38 | 0 | 25 | 2 | |
| Stem | 11 | 0 | 16 | 0 | 14 | 0 | 8 | 0 | |
| Bark | 6 | 0 | 11 | 0 | 8 | 0 | 7 | 0 | |
| Roots | 4 | 0 | 8 | 0 | 8 | 0 | 2 | 0 | |
| Total | 106 | | 106 | | 100 | | 68 | | 1174 |
| Collected | | | | | | | | | |
| Samples | | 0 | | 12 | | 0 | | 6 | 51 |
| w/CB | | | | | | | | | |
| | | | | | | | | | 5.20% |

Ooty¹: Ooty village in Tamil Nadu, Southern India
Puging²: Puging village in Upper Siang district of Arunachal Pradesh India
Singing³: Singing village in Upper Siang district of Arunachal Pradesh India
Pekong⁴: Pekong village in Upper Siang district of Arunachal Pradesh India
Mouling⁵: Mouling Natinoal Park in Arunachal Pradesh India
Kaying⁶: Kaying village West Siang district of Arunachal Pradesh India
Lipo⁷: Lipo village West Siang district of Arunachal Pradesh India
Bomdeling⁸: Bomdeling Wildlife Sanctuary in Bhutan
Nanda Devi⁹: Nanda Devi National Park in Himachal Pradesh, India

The Pekong strains were identified as having unusually high cannabidiol content, with detectable levels of cannabigerol (CBG), cannabichromene (CBC), cannabidiol (CBD), cannabielsoin (CBE) and cannabidivarin (CBDV) found. The content of cannabidiol, cannabichromene and cannabigerol was high, usually >85-90% of the carboxylated cannabinoids and >65-70% of the uncarboxylated cannabinoids. No trace amounts of tetrahydrocannabinol were detected in the Pekong strains.

Table 2 (below) summarizes the inflorescence size and cannabinoid level (milligram cannabinoid per gram of freeze dried plant tissue) of six of the H. yunnanesis plants collected from the Pekong region. Of these samples, samples 3, 4, and 6 were selected for breeding based on their high cannabinoid content. All of these samples were negative for the presence of tetrahydrocannabinol.

TABLE 2
Characteristics of Pekong H. yunnanesis

| | Pekong #1 | | Pekong #2 | | Pekong #3 | | Pekong #4 | | Pekong #5 | | Pekong #6 | |
|---|---|---|---|---|---|---|---|---|---|---|---|---|
| Inflorescence size | 3.7 | cm | 4.8 | cm | 7.4 | cm | 6.2 | cm | 5.9 | cm | 6.6 | cm |
| (length in cm) | | | | | | | | | | | | |
| Inflorescence | 22 | mg/g | 26 | mg/g | 56 | mg/g | 41 | mg/g | 32 | mg/g | 42 | mg/g |
| Cannabinoid | | | | | | | | | | | | |
| (mg/gram) | | | | | | | | | | | | |
| Leaf Cannabinoid | 6.3 | mg/g | 6.3 | mg/g | 7.5 | mg/g | 5.3 | mg/g | 6.1 | mg/g | 4.8 | mg/g |
| (mg/gram) | | | | | | | | | | | | |

To initiate the generation of ‘kriya’, Pekong #3 plant was crossed with Pekong #6 plant to produce 128 female progeny. The cannabinoid level in female inflorescence of each plant was assessed. Of the 128 progeny, 74 of the plants did not contain a significant cannabinoid level. Twenty-three of the progeny had longer inflorescence (>6 cm), but the level of cannabinoid in the inflorescence was less than 20 milligrams per gram of freeze dried tissue. Twenty-four of the progeny had medium inflorescence (between 4 and 6 cm in length) and a medium inflorescence cannabinoid level (between 25 and 35 mg/g). Seven of the offspring had inflorescence greater than 7 cm in length and a cannabinoid level greater than 70 mg/g. From this analysis it was surmised that the presence of cannabinoids is a recessive trait in Humulus yunnanensis.

In parallel with the cross of Pekong #3 and Pekong #6, Pekong #3 was independently crossed with Pekong #4 plant to generate 128 progeny. The cannabinoid level in the female inflorescence was also assessed in these progeny. Eight plants having an inflorescence cannabinoid level between 66 mg/g and 73 mg/g were identified.

First generation plants produced by the cross between Pekong #3 and Pekong #6 and the cross between Pekong #3 and Pekong #4 having the highest cannabinoid level were selected for crossbreeding to produce second generation plants (n=7 plants from the Pekong #3/Pekong #6 cross; n=8 plants from the Pekong #3/Pekong #4 cross). The offspring of these crosses were further bred to produce third and fourth generation offspring. The fifth recessive generation yielded female plants having an average cannabinoid content of 128 milligrams cannabinoid per gram of freeze dried inflorescence and 16 milligrams cannabinoid per gram of freeze dried leaves and “trims”. Of these female plants, one plant was chosen for asexual propagation. This new variety of high cannabinoid content plant was named Humulus yunnanensis var kriya or ‘kriya’.

Further propagation of female ‘kriya’ was carried out using in-vitro culture starting on Mar. 14, 2017 in Nainital, Uttarakhand, India. The hypocotyl and the newly germinating buds of the female ‘kriya’ plant were micro-propagated. Sterile plant tissues were removed from an intact plant. A small portion of plant tissue was placed on B5 medium to half its ionic strength (B5/2). The medium was thickened with agar to create a gel which supported the explant during growth.

The tissue samples produced during the first stage were multiplied to increase overall number. The tissue was grown into small “plantlets”. Offshoot production was induced by hormone treatment. All propagules of ‘kriya’ have been observed to be true to type in that during all asexual multiplication, the inflorescences and globose of the original plant have been maintained. After the formation of multiple shoots, these shoots were transferred to rooting medium with a high auxin\cytokinin ratio.

The presence of cannabinoids was most frequently detected in H. yonnanensis collected from the Pekong region. Table 2 above shows the cannabinoid levels in the six Pekong plants identified has having the highest cannabinoid levels, including the originating parent plants of ‘kriya’. Table 3 below shows average cannabinoid content in the inflorescence and leaves of first, second, third, and fourth generation² offspring of crossed Pekong samples, as well the average cannabinoid content in the inflorescence and leaves of ‘kriya’. The average cannabinoid level in ‘kriya’ inflorescence is 133.5 mg/g±8.62 mg/g. This level is >2-fold higher than the inflorescence cannabinoid level of the original Pekong parental variants (41 mg/g-56 mg/g). This level is also markedly different than the average inflorescence cannabinoid level found in first, second, third, and fourth generation plants. ²First generation plants are progeny of the cross between Pekong #3 and Pekong #6 plants, and the progeny of Pekong #3 and Pekong #4 plants. Second generation plants are progeny of Pekong #3/Pekong #6 offspring crossed with Pekong #3/Pekong #4 offspring. Third generation plants are progeny of second generation crosses, and fourth generation plants are progeny of third generation crosses.

The average cannabinoid leaf content of ‘kriya’ is 19.28±3.75 mg/g. This is also >2-fold higher than the leaf cannabinoid content of the originating Pekong parental variants (4.8 mg/g-7.5 mg/g). This level is also markedly different from the average leaf cannabinoid level found in the first, second, third, and fourth generation plants.

Cannabidiol (CBD), cannabichromene (CBC), and cannabigerol (CBG) make up >98% of the cannabinoids present in the inflorescence and leaves of ‘kriya’. Trace amounts (<2%) of cannabielsoin (CBE) and cannbidivarin (CBDV) are also present. As with the parent strains, no tetrahydrocannabinol is present in ‘kriya’.

TABLE 3
Cannabinoid Content of ‘Kriya’ and its Predecessors

| | Inflorescence Length | | | | Inflorescence Cannabinoid | | | | Leaf Cannabinoid | | | |
|---|---|---|---|---|---|---|---|---|---|---|---|---|
| | | Mean | Standard | | | Mean | Standard | | | Mean | Standard | |
| Plant | N | (cm) | Deviation | Variance | N | (mg/g) | Deviation | Variance | N | (mg/g) | Deviation | Variance |
| 1st Generation | 76 | 5.767 | 1.325 | 1.755 | 76 | 33.167 | 8.06 | 64.967 | 83 | 1.78 | 0.342 | 0.117 |
| 2nd Generation | 62 | 5.933 | 1.294 | 1.675 | 62 | 34.667 | 8.335 | 69.467 | 73 | 2.24 | 0.623 | 0.388 |
| 3rd Generation | 47 | 6.15 | 1.319 | 1.739 | 47 | 46.334 | 7.23 | 52.267 | 48 | 3.4 | 0.678 | 0.46 |
| 4th Generation | 52 | 6.783 | 0.861 | 0.742 | 52 | 78.333 | 8.123 | 66.967 | 51 | 6.48 | 1.105 | 1.222 |
| Humulus kriya | 36 | 8.233 | 0.857 | 0.735 | 36 | 133.5 | 8.62 | 74.3 | 46 | 19.28 | 3.749 | 14.057 |

Table 4 below shows average β-caryophyllene content in the inflorescence and leaves of first, second, third, and fourth generation offspring of crossed Pekong samples, as well the average β-caryophyllene content in the inflorescence and leaves of ‘kriya’. The average β-caryophyllene level in ‘kriya’ inflorescence is 53.11 mg/g±7.73 mg/g. This level is >4-fold higher than the inflorescence β-caryophyllene level of the original Pekong parental variants (3 mg/g-11 mg/g). This level is also markedly different than the average inflorescence β-caryophyllene level found in first, second, third, and fourth generation plants.

The average β-caryophyllene leaf content of ‘kriya’ is 10.63±1.99 mg/g. This is almost 10-fold higher than the leaf β-caryophyllene content of the originating Pekong parental variants (0.8 mg/g-1.6 mg/g). This level is also markedly different from the average leaf β-caryophyllene level found in the first, second, third, and fourth generation plants.

TABLE 4
β-Caryophyllene Content of ‘Kriya’ and its Predecessors

| | Inflorescence Beta Caryophyllene | | | | Leaf Beta Caryophyllene | | | |
|---|---|---|---|---|---|---|---|---|
| | | Mean | Standard | | | Mean | Standard | |
| | N | (mg/g) | Deviation | Variance | N | (mg/g) | Deviation | Variance |
| 1st Generation | 76 | 26.77 | 6.41 | 41.11 | 67 | 2.09 | 0.411 | 0.168 |
| 2nd Generation | 54 | 28.52 | 4.80 | 23.06 | 73 | 2.77 | 0.52 | 0.28 |
| 3rd Generation | 63 | 36.52 | 7.81 | 61.08 | 59 | 4.17 | 0.54 | 0.29 |
| 4th Generation | 47 | 42.42 | 8.33 | 69.54 | 71 | 7.9 | 1.87 | 3.49 |
| Humulus kriya | 73 | 53.11 | 7.73 | 59.82 | 82 | 10.63 | 1.99 | 3.98 |

### Example 2—Humulus yunnanensis kriya Extracts

A raw extract of the pod from H. yunnanensis kriya (late vegetative state) was analyzed by the Food and Safety Standards Authority of India. The composition of the extract is provided in Table 5 below.

TABLE 5
Humulus Plant Extract

| Component | % composition |
|---|---|
| 6-prenylnaringenin | 6 |
| 8-prenylnaringenin | 4 |
| adhumulone | 5 |
| adlupulone | 6 |
| cannabichromene | 5 |
| cannabidiol | 12 |
| cannabidivarin | 2 |
| cannabigerol | 2 |
| farnesene | 5 |
| humulone | 9 |
| isoxanthohumol | 6 |
| lupulone | 8 |
| xanthohumol | 7 |
| α-humulene | 9 |
| β-caryophyllene | 14 |

### Example 3—Higher Bioactivity Cannabidiol in Greater Concentration More Greatly Reduces Valvular Interstitial Cell Calcification

### Materials and Methods for Example 3

All chemicals and solutions were obtained from Sigma-Aldrich (St. Louis, Mo.). All cell cultures were obtained from Creative Bioarray, Shirley, N.Y. CBD bioactivity was measured using practices described in Cushing et al., “Measuring the Bioactivity of Phytocannabinoid Cannabidiol from Cannabis Sources, and a Novel Non-Cannabis Source,” Journal of Medical Phyto. Research 10 (2018), which is hereby incorporated by reference in its entirety.

VIC Isolation and Culture. VICs were isolated from porcine aortic valve leaflets (Hormel, Austin, Minn.) by collagenase digestion and subsequently cultured in growth medium (15% FBS, 2 mM L-glutamine, 100 U/ml penicillin, and 100 g/ml streptomycin in medium 199) at 37° C., 5% CO₂ for two to four passages. VICs used in all experiments were seeded at a density of 50,000 cells/cm² onto 24-well or 96-well plates. During the experiments, the VICs were cultured in low-serum medium (1% FBS, 100 U/ml penicillin, 100 g/ml streptomycin, 2 mM L-glutamine, in medium 199), and the medium was changed each day until the fifth day.

Culture Substrate Coatings. Tissue culture polystyrene (TCPS) plates (24-well or 96-well) were coated with type I collagen (Coll) (Inamed Biomaterials, Fremont, Calif.; 2 g/cm²), fibronectin (FN, 5 g/cm²), fibrin (FB, 1.5 g/cm²), or left untreated (TCPS). For the FB coating, plates were first incubated overnight at 4° C. in fibrinogen (1 mg/mL), followed by three washes with 0.05% Tween 20 in phosphate-buffered saline (PBS) and 1 hour incubation with thrombin (0.6 mg/ml) at 37° C. (Gu et al., “Role of the MAPK/ERK Pathway in Valvular Interstitial Cell Calcification,” American Journal of Physiology—Heart and Circulatory Physiology 296(6):H1748-H1757 (2009), which is hereby incorporated by reference in its entirety). All coatings were prepared in 50 mM bicarbonate coating buffer, pH 8.5, and rinsed three times with PBS before cell seeding. The amounts of adsorbed proteins were measured on separate plates using the bicinchoninic acid protein assay (Pierce, Rockford, Ill.) to verify adsorption of protein coatings.

MEK-1/2 Inhibition. VICs exposed to various concentrations and bioactivities of CBD were treated with U-0126 [1,4-diamino-2,3-dicyano-1,4-bis(2-aminophenylthio) butadiene; Calbiochem, San Diego, Calif.], PD-98059 (2-amino-3 methoxyflavone; 5 M; Calbiochem), or left untreated as a control to confirm the MAPK specificity of these inhibition experiments. U-0126 specifically inhibits MEK-1/2, thus inhibiting activation of ERK-1/2 (Favata et al., “Identification of a Novel Inhibitor of Mitogen-Activated Protein Kinase Kinase,” Journal of Biological Chemistry 273(29): 18623-18632 (1998), which is hereby incorporated by reference in its entirety). PD-98059 is an alternate MEK inhibitor. 9 tissue samples were in each treatment group. These were the tissue samples used in subsequent analyses.

Quantification of Cell Number. At time points of 1, 3, and 5 days, VICs were lysed with radioimmunoprecipitation assay buffer [1% sodium deoxycholate, 0.1% SDS, 1% Triton X-100, 1 mM iodoacetamide, 140 mM NaCl, 10 mM Tris HCl (pH 8.0)]. The amount of DNA in sample lysates was measured via the Quanti-iT PicoGreen assay (Invitrogen, Carlsbad, Calif.), according to the manufacturer's instructions.

Migration Assay. Migration was assayed via a modified fence method (Mann et al., “Cell Adhesion Peptides Alter Smooth Muscle Cell Adhesion, Proliferation, Migration, and Matrix Protein Synthesis on Modified Surfaces and in Polymer Scaffolds,” Journal of Biomedical Materials Research 60(1): 86-93 (2002), which is hereby incorporated by reference in its entirety), wherein VICs were seeded within 2 mm² removable silicone wells, grown to confluency, and then allowed to migrate following the detachment of silicone isolators (defined as day 0). Grid-patterned transparencies were attached underneath plates containing VIC cultures to track cell movement over time. Photomicrographs were taken of the leading edge of cell migration under 40 magnification (Olympus IX51) every 24 hours for 5 days. Net cell edge displacement was measured by overlaying time course images and then quantifying migration distance (NIH ImageJ) by measuring the advancement of the leading cell edge subtracted from the migration area recorded on day 0 within a single grid space.

Apoptosis Assay. To ensure the health of the cell samples used in the calcification experiment, apoptosis was measured using an ELISA-based HT TiterTACS Assay Kit (Trevigen, Gaithersburg, Md.), which detects DNA fragmentation. At days 1 and 5, cells were fixed in 3.7% buffered formaldehyde solution for 7 minutes, washed with PBS, and postfixed in 100% methanol for 20 minutes. Following manufacturer's instructions, the cells were permeabilized with proteinase K, quenched with 2.5% H₂O₂ in methanol, and then incubated with the labeling reaction mix (TdT,

Biotin-dNTP, unlabeled dNTP) to label breaks in DNA. Streptavidin-HRP and then TACS-Sapphire were added to the wells to detect apoptotic cells; the reaction was stopped with 2 N HCl, and absorbance was read at 450 nm.

RNA Isolation. Total RNA was isolated using TRI Reagent (Molecular Research Center, Cincinnati, Ohio), according to the manufacturer's instructions. VICs were lysed with 2001 TRI Reagent per well at 4° C. with 50 protease inhibitor cocktail (BD Biosciences, San Jose, Calif.). The homogenate was stored at room temperature for 5 minutes to complete the dissociation of nucleoprotein complexes, at which point 0.15 mL chloroform per 600 L TRI Reagent was added to the homogenate, followed by centrifugation at 13,000 g for 15 minutes. After centrifugation, RNA was precipitated from the upper aqueous phase by adding 0.3 mL isopropanol per 600 L TRI

Reagent to the tubes and then centrifuged at 13,000 g for 8 minutes. After this centrifugation step, the RNA pellet was washed with 75% ethanol and centrifuged at 8,000 g for 5 minutes. The RNA pellet was air dried and dissolved in 75 L H₂O at 60° C. for 15 minutes. RNA samples were stored at 20° C. until subsequent use.

Quantitative Real-Time PCR Analysis. Custom primers for various markers of cell contractility and osteogenic activity were obtained from Invitrogen (Carlsbad, Calif.) and are listed in Table 6.

TABLE 6
Calcification reduction by CBD mg concentration and bioactivity.
All calcification reductions are presented as percentages.

| Concentration | | Bioactivity | | | | | | | |
|---|---|---|---|---|---|---|---|---|---|
| (mg) | Measure | .20 | .30 | .50 | .60 | .70 | .80 | .90 | .95 |
| 5 | M | 5.17 | 6.16 | 7.63 | 7.99 | 10.06 | 14.32 | 18.06 | 19.42 |
| | SD | 0.83 | 0.54 | 0.88 | 0.85 | 1.28 | 1.35 | 1.22 | 1.8 |
| | Min | 3.8 | 5.2 | 5.3 | 6.2 | 7.3 | 11.3 | 14.6 | 15.4 |
| | Max | 6.9 | 7.3 | 9.1 | 10.4 | 13.6 | 16.6 | 21 | 22.1 |
| 10 | M | 4.95 | 6.24 | 9.77 | 11.8 | 14.33 | 20.03 | 25.04 | 25.56 |
| | SD | 0.62 | 0.66 | 0.99 | 1.1 | 1.48 | 2.5 | 2.62 | 3.04 |
| | Min | 3.7 | 4.9 | 7.7 | 9.2 | 10.2 | 15.9 | 20.5 | 18.5 |
| | Max | 6.2 | 7.5 | 11.7 | 14.3 | 17.2 | 24.6 | 30.3 | 32.5 |
| 25 | M | 6.07 | 6.23 | 10.89 | 13.75 | 16.55 | 24.94 | 30.51 | 32.69 |
| | SD | 0.54 | 0.89 | 0.8 | 0.95 | 1.34 | 2.63 | 3.02 | 4.33 |
| | Min | 4.8 | 4.6 | 9.5 | 10.5 | 14.3 | 20.2 | 23.5 | 26.3 |
| | Max | 7.1 | 8.1 | 12.5 | 15.3 | 18.6 | 30.3 | 35.6 | 42.6 |
| 40 | M | 5.19 | 7.13 | 14.2 | 19.23 | 24.7 | 28.54 | 40.09 | 42.98 |
| | SD | 0.75 | 0.76 | 1.4 | 1.5 | 3.2 | 3.02 | 2.77 | 4.8 |
| | Min | 3.6 | 5.1 | 12 | 168 | 18.9 | 22.4 | 33.7 | 35.8 |
| | Max | 6.9 | 8.1 | 17.1 | 21.9 | 33 | 34.6 | 44.6 | 54.5 |
| 100 | M | 6.1 | 7.2 | 16.1 | 21.54 | 30.71 | 37.74 | 48.52 | 55 |
| | SD | 0.58 | 0.64 | 1.05 | 1.22 | 2.18 | 2.87 | 4.27 | 6.66 |
| | Min | 5.2 | 6 | 14.1 | 18.3 | 25.8 | 31.7 | 37.6 | 41.9 |
| | Max | 7.5 | 8.4 | 18.1 | 23.7 | 35.6 | 44.4 | 54.7 | 66.1 |

For cDNA construction, 250 ng of original RNA isolated from samples were reverse transcribed using iScript (Bio-Rad Laboratories, Hercules, Calif.) as per manufacturer's instructions. Samples were processed for real-time PCR analysis by combining 0.5 L of the cDNA construction, 5 M of primers, and SYBR Green SuperMix (Bio-Rad) in a 15-L reaction, as specified in the manufacturer's protocol. For thermo cycling, a standard protocol was used: PCR reactions were run over 40 cycles of denaturing at 95° C. for 15 seconds and annealed at 60° C. for 1 minute; this was followed by a melting curve analysis for 80 cycles of 55° C. 0.5° C./cycle, 10 seconds per cycle, to further confirm the purity of the final PCR products, with each condition performed in triplicate (iCycler iQ Real-Time PCR Instrument, Bio-Rad). A standard comparative threshold cycle (or CT) method was used to analyze the PCR data. The CT of all samples were first normalized to actin as an internal control, and then the CT values for experimental samples were further normalized to the negative control (VICs on Coll, which represented a non CBD condition).

Quantification of Nodule Number and Size. After 5 days of culture in the presence or absence of U-0126 or PD-98059, VIC cultures were stained with Alizarin Red S (ARS) to facilitate quantification of calcified nodules, as ARS stains mineralized deposits red. Cultures were fixed with 10% neutral buffered formalin, stored at 4° C. overnight, and stained with a 2% solution of ARS in PBS. Positively stained nodules were manually counted under a microscope (Olympus IX51 with Hamamatsu 285 digital camera and Simple PCI digital imaging software; Compix, Imaging Systems, Cranberry Township, Pa.). Nodule size was measured using ImageJ software (National Institutes of Health), and photomicrographs were captured under 40 and 100 magnifications.

CBD Samples and Bioactivity Testing. CBD samples with bioactivities 0.20, 0.30, 0.50, 0.60, and 0.70 were obtained from Randy Kindred, Natural Hemp Solutions. CBD samples with bioactivities 0.80, 0.90, and 0.95 were isolated from ImmunAG, a Humulus product of ImmunAG LLP. Following isolation, bioactivity was measured using procedures outlined in Cushing et al., “Measuring the Bioactivity of Phytocannabinoid Cannabidiol from Cannabis Sources, and a Novel Non-Cannabis Source,” Journal of Medical Phyto. Research 10 (2018), which is hereby incorporated by reference in its entirety.

### Results

Multiplate wells containing calcifying VICs from 27 different tissue samples were treated with CBD of 8 different bioactivities, and 5 different mg concentrations, or left untreated. Average calcification was computed using the number of nodules per well, and the average area per nodule. Average total nodule area for untreated wells was 3.27 mm² (SD=0.32, min=0.5, max=4.0) per well. All reported percent reductions in calcification were computed by dividing average nodule area of treated wells from 3.27 mm². CBD samples were clustered according to their bioactivity levels around the values, 0.20, 0.30, 0.50, 0.60, 0.70, 0.80, 0.90, and 0.95. Mg dosages of 5, 10, 25, 40, and 100 were examined.

Exploratory Data Analysis. Means, standard deviations, and minimum and maximum values for every tested bioactivity level at every tested mg concentration are provided in Table 6 supra. All data are provided in Table 7.

TABLE 7
All VIC calcification reduction data.

| Concen- | | Sample | Bioactivity | | | | | | | |
|---|---|---|---|---|---|---|---|---|---|---|
| tration | | ID | .20 | .30 | .50 | .60 | .70 | .80 | .90 | .95 |
| 5 | mg | 1548 | 4.2 | 5.5 | 10.4 | 8.7 | 8.6 | 13.0 | 18.5 | 21.9 |
| 5 | mg | 1730 | 4.8 | 5.7 | 8.2 | 8.2 | 13.6 | 13.8 | 19.3 | 19.2 |
| 5 | mg | 1194 | 4.7 | 5.9 | 9.1 | 7.4 | 11.6 | 16.4 | 17.8 | 21.4 |
| 5 | mg | 1785 | 5.5 | 6.4 | 7.8 | 8.1 | 11.1 | 13.1 | 21.0 | 21.2 |
| 5 | mg | 1772 | 6.9 | 6.9 | 7.5 | 7.0 | 10.7 | 15.1 | 18.0 | 18.9 |
| 5 | mg | 1169 | 3.8 | 6.1 | 7.1 | 9.1 | 10.5 | 15.3 | 17.9 | 19.7 |
| 5 | mg | 1245 | 4.1 | 5.6 | 7.8 | 7.3 | 9.9 | 14.2 | 16.0 | 19.8 |
| 5 | mg | 1496 | 4.1 | 6.4 | 7.9 | 8.8 | 10.3 | 13.8 | 14.6 | 19.4 |
| 5 | mg | 1381 | 6.1 | 6.7 | 7.6 | 5.3 | 10.7 | 13.9 | 18.1 | 20.5 |
| 5 | mg | 1380 | 4.4 | 5.5 | 8.2 | 6.3 | 10.7 | 13.6 | 18.6 | 16.7 |
| 5 | mg | 1230 | 6.3 | 5.3 | 6.2 | 7.4 | 7.3 | 16.0 | 17.2 | 21.7 |
| 5 | mg | 1655 | 5.0 | 6.1 | 10.0 | 7.2 | 11.2 | 15.0 | 17.7 | 18.8 |
| 5 | mg | 1175 | 5.4 | 6.2 | 7.4 | 7.7 | 11.6 | 11.3 | 17.7 | 19.8 |
| 5 | mg | 1617 | 6.4 | 6.3 | 7.8 | 7.4 | 8.0 | 14.0 | 18.5 | 17.6 |
| 5 | mg | 1042 | 6.4 | 6.3 | 7.5 | 7.6 | 9.5 | 14.1 | 19.1 | 18.5 |
| 5 | mg | 1640 | 5.3 | 6.0 | 8.3 | 6.2 | 9.1 | 14.6 | 16.5 | 18.8 |
| 5 | mg | 1082 | 4.3 | 5.3 | 8.1 | 7.5 | 11.0 | 14.6 | 19.0 | 16.5 |
| 5 | mg | 1743 | 5.2 | 7.3 | 7.9 | 8.1 | 9.7 | 15.9 | 19.2 | 18.5 |
| 5 | mg | 1278 | 5.3 | 6.5 | 8.2 | 8.9 | 10.1 | 15.4 | 17.8 | 22.1 |
| 5 | mg | 1134 | 4.3 | 6.4 | 8.1 | 7.5 | 9.7 | 14.6 | 18.7 | 20.4 |
| 5 | mg | 1452 | 5.4 | 5.2 | 7.8 | 7.6 | 9.5 | 16.5 | 17.5 | 16.6 |
| 5 | mg | 1696 | 5.5 | 6.4 | 7.7 | 7.8 | 8.8 | 16.6 | 19.5 | 19.7 |
| 5 | mg | 1765 | 6.0 | 5.8 | 8.7 | 7.5 | 9.6 | 13.3 | 18.1 | 18.7 |
| 5 | mg | 1032 | 5.7 | 6.3 | 7.8 | 6.8 | 10.5 | 12.4 | 17.8 | 21.1 |
| 5 | mg | 1850 | 4.5 | 6.6 | 7.7 | 7.3 | 9.5 | 13.1 | 18.7 | 21.9 |
| 5 | mg | 2018 | 4.6 | 6.5 | 6.8 | 8.8 | 10.3 | 14.7 | 17.0 | 15.4 |
| 5 | mg | 1205 | 5.4 | 7.0 | 8.2 | 8.5 | 8.5 | 12.3 | 17.8 | 19.6 |
| 10 | mg | 1548 | 3.7 | 6.6 | 8.8 | 12.7 | 15.0 | 17.5 | 23.1 | 32.5 |
| 10 | mg | 1730 | 4.6 | 6.4 | 11.2 | 11.6 | 14.4 | 19.1 | 20.5 | 18.5 |
| 10 | mg | 1194 | 4.5 | 6.5 | 10.0 | 11.4 | 13.5 | 22.8 | 20.9 | 24.2 |
| 10 | mg | 1785 | 5.9 | 6.5 | 9.3 | 11.9 | 12.8 | 18.9 | 26.2 | 27.9 |
| 10 | mg | 1772 | 4.7 | 5.3 | 10.2 | 10.6 | 16.0 | 21.7 | 24.5 | 26.8 |
| 10 | mg | 1169 | 4.7 | 5.3 | 9.1 | 12.1 | 15.7 | 16.2 | 29.6 | 24.0 |
| 10 | mg | 1245 | 4.9 | 5.6 | 9.1 | 9.2 | 13.6 | 17.1 | 23.5 | 20.8 |
| 10 | mg | 1496 | 5.1 | 7.1 | 10.8 | 14.3 | 10.2 | 18.8 | 28.3 | 31.0 |
| 10 | mg | 1381 | 4.9 | 5.9 | 9.2 | 12.7 | 12.4 | 16.4 | 22.4 | 27.9 |
| 10 | mg | 1380 | 3.9 | 6.6 | 9.2 | 11.2 | 15.5 | 19.5 | 24.7 | 25.4 |
| 10 | mg | 1230 | 5.4 | 7.0 | 10.9 | 11.6 | 15.1 | 22.5 | 23.2 | 25.1 |
| 10 | mg | 1655 | 5.9 | 5.9 | 9.5 | 10.8 | 15.6 | 22.9 | 28.6 | 27.0 |
| 10 | mg | 1175 | 4.5 | 6.7 | 9.9 | 11.4 | 13.3 | 22.0 | 25.4 | 22.6 |
| 10 | mg | 1617 | 6.2 | 6.1 | 10.6 | 11.4 | 14.4 | 20.9 | 23.8 | 28.1 |
| 10 | mg | 1042 | 4.5 | 6.6 | 11.4 | 11.5 | 15.3 | 21.9 | 21.5 | 27.1 |
| 10 | mg | 1640 | 4.6 | 6.8 | 10.6 | 11.9 | 14.0 | 18.6 | 26.9 | 22.3 |
| 10 | mg | 1082 | 5.0 | 5.2 | 8.6 | 10.8 | 17.2 | 22.7 | 23.1 | 28.7 |
| 10 | mg | 1743 | 3.7 | 4.9 | 7.7 | 13.1 | 13.5 | 24.1 | 24.5 | 24.3 |
| 10 | mg | 1278 | 5.2 | 5.3 | 11.7 | 14.0 | 16.9 | 20.2 | 24.4 | 23.5 |
| 10 | mg | 1134 | 5.2 | 6.7 | 10.0 | 11.9 | 15.0 | 16.0 | 24.0 | 25.7 |
| 10 | mg | 1452 | 5.2 | 6.2 | 9.1 | 11.4 | 14.5 | 15.9 | 24.4 | 26.3 |
| 10 | mg | 1696 | 4.8 | 6.5 | 9.2 | 10.4 | 13.5 | 24.6 | 29.6 | 24.8 |
| 10 | mg | 1765 | 5.1 | 6.1 | 9.1 | 12.6 | 13.9 | 20.7 | 25.6 | 27.2 |
| 10 | mg | 1032 | 5.2 | 7.5 | 10.2 | 12.7 | 12.6 | 20.3 | 26.0 | 27.4 |
| 10 | mg | 1850 | 5.8 | 6.2 | 10.8 | 12.6 | 13.4 | 19.0 | 27.0 | 21.3 |
| 10 | mg | 2018 | 5.0 | 5.9 | 8.8 | 12.3 | 14.1 | 20.8 | 24.0 | 25.6 |
| 10 | mg | 1205 | 5.4 | 7.1 | 8.8 | 10.6 | 15.4 | 19.8 | 30.3 | 24.1 |
| 25 | mg | 1548 | 5.6 | 5.5 | 11.9 | 15.3 | 16.3 | 28.5 | 31.6 | 34.9 |
| 25 | mg | 1730 | 5.9 | 7.1 | 10.3 | 13.1 | 14.8 | 27.7 | 34.4 | 27.7 |
| 25 | mg | 1194 | 6.8 | 6.9 | 11.8 | 14.6 | 16.3 | 22.4 | 31.6 | 34.0 |
| 25 | mg | 1785 | 5.7 | 6.6 | 10.7 | 12.7 | 14.7 | 22.7 | 28.3 | 29.2 |
| 25 | mg | 1772 | 6.3 | 6.7 | 11.1 | 13.5 | 18.4 | 20.2 | 31.7 | 34.4 |
| 25 | mg | 1169 | 5.2 | 6.1 | 10.5 | 14.2 | 18.6 | 27.2 | 31.5 | 30.3 |
| 25 | mg | 1245 | 6.0 | 7.2 | 10.4 | 13.1 | 17.0 | 29.9 | 25.5 | 34.8 |
| 25 | mg | 1496 | 5.5 | 8.1 | 11.0 | 13.5 | 17.0 | 25.9 | 30.1 | 31.6 |
| 25 | mg | 1381 | 5.8 | 6.1 | 11.5 | 13.8 | 15.0 | 23.3 | 35.1 | 31.4 |
| 25 | mg | 1380 | 6.3 | 4.9 | 10.0 | 14.6 | 15.5 | 25.0 | 29.1 | 42.4 |
| 25 | mg | 1230 | 5.6 | 7.6 | 10.2 | 13.1 | 18.4 | 27.7 | 33.1 | 29.7 |
| 25 | mg | 1655 | 7.1 | 4.9 | 12.1 | 13.4 | 15.8 | 28.2 | 30.8 | 42.6 |
| 25 | mg | 1175 | 6.9 | 6.8 | 10.9 | 14.3 | 15.6 | 23.9 | 29.1 | 32.3 |
| 25 | mg | 1617 | 5.7 | 4.6 | 9.8 | 12.8 | 18.0 | 24.6 | 28.4 | 35.6 |
| 25 | mg | 1042 | 6.1 | 7.0 | 11.9 | 10.5 | 17.8 | 30.3 | 23.5 | 34.1 |
| 25 | mg | 1640 | 5.7 | 5.0 | 11.1 | 14.0 | 16.3 | 25.5 | 32.9 | 31.0 |
| 25 | mg | 1082 | 6.4 | 6.6 | 11.4 | 13.3 | 17.9 | 24.6 | 27.9 | 30.4 |
| 25 | mg | 1743 | 6.2 | 5.6 | 11.4 | 14.6 | 14.3 | 22.4 | 26.8 | 29.7 |
| 25 | mg | 1278 | 5.4 | 6.9 | 10.7 | 13.3 | 14.6 | 24.2 | 33.7 | 39.3 |
| 25 | mg | 1134 | 6.7 | 6.4 | 11.5 | 15.0 | 17.9 | 22.6 | 27.5 | 34.3 |
| 25 | mg | 1452 | 6.5 | 6.9 | 10.6 | 14.2 | 17.6 | 23.1 | 27.1 | 38.9 |
| 25 | mg | 1696 | 4.8 | 6.2 | 11.3 | 14.2 | 16.9 | 23.2 | 35.6 | 28.6 |
| 25 | mg | 1765 | 6.4 | 5.6 | 12.5 | 14.2 | 16.6 | 26.5 | 29.9 | 32.2 |
| 25 | mg | 1032 | 6.4 | 5.4 | 9.8 | 13.2 | 18.3 | 25.2 | 30.4 | 31.9 |
| 25 | mg | 1850 | 6.2 | 6.3 | 10.4 | 13.7 | 16.7 | 24.9 | 31.0 | 26.5 |
| 25 | mg | 2018 | 6.3 | 5.3 | 9.7 | 14.8 | 15.0 | 20.6 | 33.2 | 28.6 |
| 25 | mg | 1205 | 6.4 | 5.9 | 9.5 | 14.3 | 15.6 | 23.1 | 33.9 | 26.3 |
| 40 | mg | 1548 | 3.8 | 7.9 | 12.4 | 21.9 | 30.0 | 32.3 | 40.6 | 39.6 |
| 40 | mg | 1730 | 5.2 | 7.8 | 16.0 | 18.1 | 28.2 | 27.6 | 34.7 | 43.0 |
| 40 | mg | 1194 | 5.3 | 7.6 | 12.1 | 20.4 | 24.6 | 32.8 | 42.1 | 47.9 |
| 40 | mg | 1785 | 4.9 | 7.8 | 13.2 | 20.2 | 24.7 | 34.6 | 44.6 | 42.6 |
| 40 | mg | 1772 | 5.2 | 7.5 | 13.9 | 17.3 | 24.8 | 23.4 | 37.5 | 47.7 |
| 40 | mg | 1169 | 4.7 | 5.8 | 17.1 | 18.0 | 29.7 | 30.6 | 39.6 | 46.8 |
| 40 | mg | 1245 | 4.6 | 6.6 | 14.9 | 19.8 | 25.7 | 29.7 | 38.1 | 47.1 |
| 40 | mg | 1496 | 4.7 | 8.0 | 14.9 | 21.2 | 19.9 | 29.1 | 39.4 | 44.8 |
| 40 | mg | 1381 | 4.4 | 6.0 | 13.2 | 17.6 | 22.1 | 29.0 | 38.5 | 46.7 |
| 40 | mg | 1380 | 6.3 | 7.7 | 14.7 | 16.8 | 24.3 | 27.9 | 42.4 | 36.5 |
| 40 | mg | 1230 | 5.4 | 6.9 | 16.5 | 19.2 | 33.0 | 29.4 | 41.5 | 36.7 |
| 40 | mg | 1655 | 5.5 | 6.2 | 15.4 | 18.6 | 22.6 | 29.2 | 43.1 | 43.0 |
| 40 | mg | 1175 | 3.6 | 7.3 | 13.8 | 20.4 | 24.9 | 26.7 | 36.2 | 38.5 |
| 40 | mg | 1617 | 4.5 | 6.9 | 13.3 | 19.3 | 18.9 | 22.4 | 42.3 | 51.1 |
| 40 | mg | 1042 | 5.0 | 6.7 | 12.6 | 21.7 | 25.9 | 33.9 | 43.1 | 41.6 |
| 40 | mg | 1640 | 4.7 | 8.1 | 13.2 | 17.1 | 22.5 | 26.2 | 42.1 | 46.1 |
| 40 | mg | 1082 | 5.3 | 7.7 | 15.0 | 19.7 | 22.8 | 29.5 | 38.8 | 37.9 |
| 40 | mg | 1743 | 6.2 | 6.8 | 13.9 | 20.2 | 24.3 | 25.3 | 36.0 | 36.9 |
| 40 | mg | 1278 | 5.9 | 8.1 | 15.2 | 17.6 | 19.8 | 23.6 | 40.2 | 44.1 |
| 40 | mg | 1134 | 5.7 | 7.0 | 14.2 | 21.6 | 24.4 | 27.0 | 39.4 | 42.1 |
| 40 | mg | 1452 | 5.2 | 7.2 | 14.2 | 19.7 | 23.9 | 26.5 | 40.1 | 39.2 |
| 40 | mg | 1696 | 6.0 | 6.8 | 12.0 | 17.9 | 25.3 | 27.6 | 39.6 | 35.8 |
| 40 | mg | 1765 | 6.9 | 8.0 | 14.8 | 17.4 | 23.2 | 30.6 | 41.9 | 39.6 |
| 40 | mg | 1032 | 5.2 | 6.6 | 15.2 | 20.6 | 25.5 | 31.6 | 33.7 | 38.9 |
| 40 | mg | 1850 | 5.0 | 5.1 | 16.3 | 18.8 | 27.4 | 27.9 | 42.8 | 44.1 |
| 40 | mg | 2018 | 6.1 | 7.0 | 12.6 | 18.5 | 21.4 | 27.8 | 43.4 | 47.6 |
| 40 | mg | 1205 | 4.7 | 7.3 | 12.7 | 19.6 | 27.2 | 28.4 | 40.7 | 54.5 |
| 100 | mg | 1548 | 6.0 | 7.9 | 16.1 | 23.7 | 30.2 | 41.6 | 51.8 | 52.5 |
| 100 | mg | 1730 | 6.5 | 6.9 | 16.0 | 19.9 | 26.8 | 38.8 | 51.9 | 43.0 |
| 100 | mg | 1194 | 5.3 | 8.4 | 16.5 | 21.6 | 35.6 | 40.1 | 49.3 | 59.8 |
| 100 | mg | 1785 | 5.8 | 6.9 | 14.9 | 21.7 | 31.6 | 35.8 | 47.4 | 55.6 |
| 100 | mg | 1772 | 6.1 | 7.4 | 17.9 | 20.7 | 30.5 | 39.2 | 53.8 | 59.0 |
| 100 | mg | 1169 | 5.4 | 7.3 | 14.8 | 21.7 | 29.7 | 44.4 | 47.6 | 50.7 |
| 100 | mg | 1245 | 6.1 | 8.1 | 15.3 | 22.6 | 32.2 | 39.2 | 49.8 | 51.4 |
| 100 | mg | 1496 | 5.8 | 6.7 | 14.1 | 21.4 | 29.4 | 33.8 | 53.6 | 41.9 |
| 100 | mg | 1381 | 6.5 | 7.7 | 15.7 | 22.1 | 29.3 | 35.6 | 51.6 | 60.4 |
| 100 | mg | 1380 | 6.5 | 6.7 | 16.4 | 20.5 | 30.4 | 39.6 | 45.2 | 62.0 |
| 100 | mg | 1230 | 6.8 | 7.6 | 16.4 | 18.3 | 29.6 | 38.2 | 44.9 | 66.1 |
| 100 | mg | 1655 | 6.5 | 6.9 | 15.0 | 22.5 | 31.9 | 36.3 | 50.3 | 59.6 |
| 100 | mg | 1175 | 5.7 | 6.5 | 15.9 | 22.5 | 31.1 | 36.1 | 44.4 | 63.2 |
| 100 | mg | 1617 | 5.2 | 7.8 | 18.1 | 21.8 | 31.1 | 37.1 | 45.0 | 51.5 |
| 100 | mg | 1042 | 6.2 | 6.7 | 16.5 | 21.6 | 27.7 | 35.0 | 53.9 | 57.0 |
| 100 | mg | 1640 | 6.6 | 6.0 | 17.1 | 20.5 | 30.4 | 39.7 | 38.3 | 64.7 |
| 100 | mg | 1082 | 5.5 | 6.5 | 15.5 | 21.3 | 31.5 | 31.7 | 54.7 | 58.4 |
| 100 | mg | 1743 | 6.4 | 7.1 | 16.7 | 20.7 | 29.6 | 41.8 | 46.6 | 56.7 |
| 100 | mg | 1278 | 7.4 | 7.4 | 15.8 | 21.8 | 32.0 | 39.5 | 37.6 | 41.9 |
| 100 | mg | 1134 | 5.9 | 7.5 | 14.3 | 19.3 | 31.5 | 39.7 | 46.0 | 51.9 |
| 100 | mg | 1452 | 5.8 | 7.3 | 17.3 | 22.8 | 33.1 | 37.2 | 51.5 | 43.6 |
| 100 | mg | 1696 | 7.5 | 8.4 | 15.7 | 22.0 | 35.0 | 38.5 | 51.8 | 56.6 |
| 100 | mg | 1765 | 5.8 | 7.6 | 15.4 | 22.4 | 32.2 | 35.9 | 47.4 | 56.3 |
| 100 | mg | 1032 | 6.5 | 6.2 | 17.3 | 22.4 | 27.9 | 36.5 | 47.9 | 59.3 |
| 100 | mg | 1850 | 5.7 | 6.7 | 17.4 | 23.3 | 31.5 | 39.9 | 47.5 | 52.0 |
| 100 | mg | 2018 | 5.6 | 6.6 | 15.5 | 20.3 | 31.7 | 34.8 | 50.3 | 53.6 |
| 100 | mg | 1205 | 5.6 | 7.6 | 17.2 | 22.3 | 25.8 | 33.1 | 50.0 | 56.4 |

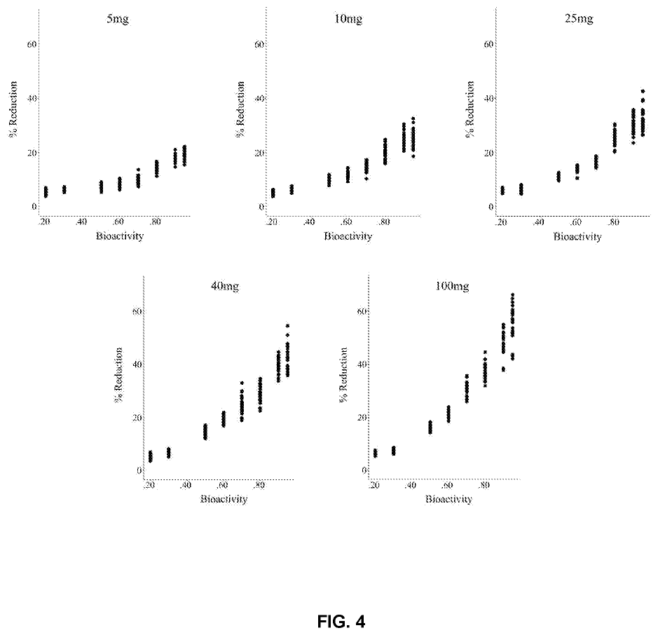

Higher bioactivities and higher concentrations each corresponded with reductions in calcification. At all five concentrations, with increases in bioactivity, calcification appeared to reduce exponentially (See FIG. 4). Variance between the calcification reductions of each sample also increased as bioactivity increased. For example, the group with the highest reduction values (100 mg, 0.95 bioactivity) showed between 40% and 67% reduction (M=55%). This group also had the greatest standard deviation (SD=6.66%). Despite increased variability at higher reduction scores, meaningful differences appeared to exist between bioactivity levels. For example, for every mg concentration, the minimum calcification reduction elicited by 0.95 bioactivity CBD was still greater than the maximum calcification reduction elicited by 0.70 bioactivity CBD.

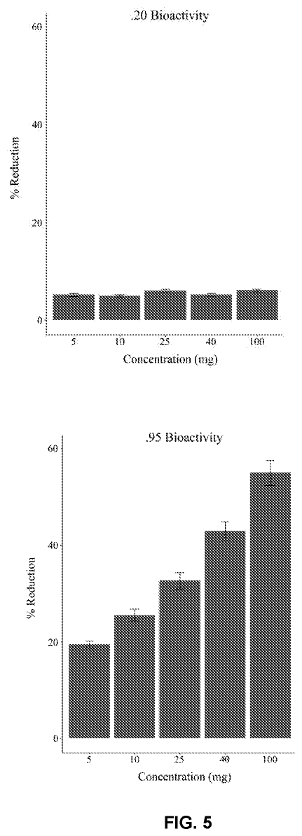

Comparing Low Bioactivity CBD Across mg Concentrations. A 1×5 repeated measures ANOVA was conducted to examine whether differences existed between the various mg concentrations (5, 10, 25, 40, 100) among VIC samples treated with 0.20 bioactivity CBD (See FIG. 5). Mauchley's Test found no violation to the assumption of sphericity between samples, W=0.759, p=0.667. The RM ANOVA found a significant difference among dosages on VIC calcification reduction within the 0.20 Bioactivity test group, F(4, 104)=18.90,p<0.001, η²G=0.354. Post-hoc comparisons using the Bonferroni correction indicated that test samples exposed to dosages of 25 mg and 100 mg showed more reduction in VIC calcification than dosages 5, 10, and 40 mg. In all significant post-hoc comparisons, p<0.001. The differences between these means were small (all M_{diff}<1.15%).

Comparing High Bioactivity CBD Across mg Concentrations. A 1×5 repeated measures ANOVA was conducted to examine whether differences existed between the various mg concentrations among VIC samples treated with 0.95 bioactivity CBD (See FIG. 5). Mauchley's Test found a possible violation to the assumption of sphericity, W=0.331,p=0.0014. A repeated measures ANOVA with a Greenhouse-Geisser correction found a significant difference among dosages on VIC calcification within the 0.95 bioactivity test group, F(4, 104)=260.13,p<0.001 η²G=0.894. Post-hoc comparisons using the Bonferroni adjustment indicated significant differences across every group. In all significant post hoc comparisons, p<0.0000001. The differences between these means were much larger (largest M_{diff}=35.58%).

Examining the Interaction Between Bioactivity and Concentration. Since there was an obvious trend across the data showing that higher bioactivity and concentration each lead to greater reductions in calcification, a 2×2 mixed ANOVA was attempted to better understand the degree to which bioactivity and dosage interact. Since the increase in calcification reduction appeared to occur similarly across all mg concentrations, the statistical approach was simplified by examining only the lowest and highest bioactivities and concentrations.

Levene's test showed heterogeneous variances, W=23.42,p<0.001. Arcsine transformation was unable to render the variances homogeneous, W=16.04,p<0.001. Thus, a Robust 2×2 ANOVA using a median estimator was conducted on the original values. It found significant effects of bioactivity as a main effect (psihat=64.7, p<0.001), of concentration as a main effect (psihat=−37.5, p<0.001), and of an interaction between bioactivity and concentration (psihat=−36.5,p<0.001).

### Conclusions

At high bioactivities and concentrations, calcification was reduced greatly. At lower bioactivities, mg concentration played an inconsistent role in calcification reduction. At high bioactivities, calcification reduction increased significantly at higher concentrations. A significant interaction effect was found between bioactivity and concentration through a robust estimation technique. Taken together, these results suggest that the bioactivity of the CBD is of central importance when considering the efficacy of CBD as a VIC calcification treatment.

### Discussion

In this study, CBD was shown to be effective at reducing porcine VIC calcification in vitro, with the highest bioactivity CBD at the highest concentration showing the greatest reductions (M=55%).

Within this experiment, it was found that ERK inhibition is the most likely mechanism by which CBD chiefly reduced VIC calcification (see Gu et al., “Role of the MAPK/ERK Pathway in Valvular Interstitial Cell Calcification,” American Journal of Physiology—Heart and Circulatory Physiology 296(6):H1748-H1757 (2009), which is hereby incorporated by reference in its entirety). If this judgment is correct, then the differential calcification reduction caused by bioactivity level implies the differential inhibition of ERK activity. The implications of this are vast.

The ERK pathway links diverse extracellular stimuli to proliferation, differentiation, survival, and vascularization (Roy et al., “Phenotypic Modulation of Arterial Smooth Muscle Cells is Associated with Prolonged Activation of ERK1/2,” Differentiation 67(1-2):50-58 (2001); Salasznyk et al., “ERK Signaling Pathways Regulate the Osteogenic Differentiation of Human Mesenchymal Stem Cells on Collagen I and Vitronectin,” Cell Communication & Adhesion 11(5-6):137-153 (2004); Lewis et al., “Signal Transduction through MAP Kinase Cascades,” In Advances in Cancer Research 74:49-139 (1998). Academic Press.; Depeille, “MKK signaling and vascularization,” Oncogene 26(9):1290-6 (2007), which are hereby incorporated by reference in their entirety). A tremendous number of diseases can be affected through its modulation, including cancers (e.g., Wagner et al., “Signal Integration by JNK and p38 MAPK Pathways in Cancer Development,” Nature Reviews Cancer 9(8):537 (2009); Huang et al., “Inhibition of MAPK Kinase Signaling Pathways Suppressed Renal Cell Carcinoma Growth and Angiogenesis in Vivo,” Cancer Research 68(1):81-88 (2008); Herrera et al., “MAPK is Involved in CB2 Receptor-Induced Apoptosis of Human Leukaemia Cells,” FEBS Letters 579(22):5084-5088 (2005); Milella et al., “Therapeutic Targeting of the MEK/MAPK Signal Transduction Module in Acute Myeloid Leukemia,” The Journal of Clinical Investigation 108(6):851-859 (2001), which are hereby incorporated by reference in their entirety). For example, in conjunction with radiation, the MAPK p38 pathway was one of the main drivers of CBD-induced cell death in Glioblastoma (Ivanov et al., “Regulation of Human Glioblastoma Cell Death by Combined Treatment of Cannabidiol, γ-Radiation and Small Molecule Inhibitors of Cell Signaling Pathways,” Oncotarget 8(43):74068 (2017), which is hereby incorporated by reference in its entirety). Additionally, the modulation by CBD of ERK and ROS pathways lead to the down-regulation of Id-1 expression and the up-regulation of Id-2, thereby inhibiting breast cancer cell proliferation and invasion (McAllister et al., Pathways Mediating the Effects of Cannabidiol on the Reduction of Breast Cancer Cell Proliferation, Invasion, and Metastasis,” Breast Cancer Res. Treat. 129(1):37-47 (2011), which is hereby incorporated by reference in its entirety). The degree to which CBD elicits these anti-cancer effects would as well depend on the bioactivity of the CBD.

The bioactivity testing procedure used in this experiment, as described in Cushing et al., “Measuring the Bioactivity of Phytocannabinoid Cannabidiol from Cannabis Sources, and a Novel Non-Cannabis Source,” Journal of Medical Phyto. Research 10 (2018), which is hereby incorporated by reference in its entirety, consisted of a monoclonal antibody test whose validity was based on the CB2 affinity of CBD samples. It is as of yet unclear whether the CBD bioactivity test predicts the calcification reduction effects of non-CB2 targets, such as GPR55 (Lauckner et al., “GPR55 is a Cannabinoid Receptor that Increases Intracellular Calcium and Inhibits M Current,” Proceedings of the National Academy of Sciences 105(7):2699-2704 (2008), which is hereby incorporated by reference in its entirety). It is expected that bioactivity generalizes to effects that are mediated through non-CB2 pathways. If so, research that utilized low bioactivity CBD to explore its pro-calcific effects on pathways such as GPR55 may have produced erroneous results. It is imperative that CBD samples be tested for bioactivity prior to clinical research.

### Example 4—Bioactive Cannabidiol More Greatly Reduces Valvular Interstitial Cell Calcification When Combined with β-Caryophyllene, and α-Humulene

### Materials and Methods for Example 4

This study was carried out concurrently with the study described supra. All chemicals and solutions were obtained from Sigma-Aldrich (St. Louis, Mo.). All cell cultures were obtained from Creative Bioarray, Shirley, N.Y. CBD bioactivity was measured using practices described in Cushing et al., “Measuring the Bioactivity of Phytocannabinoid Cannabidiol from Cannabis Sources, and a Novel Non-Cannabis Source,” Journal of Medical Phyto. Research 10 (2018), which is hereby incorporated by reference in its entirety.

VIC Isolation and Culture. VICs were isolated from porcine aortic valve leaflets (Hormel, Austin, Minn.) by collagenase digestion and subsequently cultured in growth medium (15% FBS, 2 mM L-glutamine, 100 U/ml penicillin, and 100 g/ml streptomycin in medium 199) at 37° C., 5% CO₂ for two to four passages. VICs used in all experiments were seeded at a density of 50,000 cells/cm² onto 24-well or 96-well plates. During the experiments, the VICs were cultured in low-serum medium (1% FBS, 100 U/ml penicillin, 100 g/ml streptomycin, 2 mM L-glutamine, in medium 199), and the medium was changed each day until the fifth day.

Culture Substrate Coatings. Tissue culture polystyrene (TCPS) plates (24-well or 96-well) were coated with type I collagen (Coll) (Inamed Biomaterials, Fremont, Calif.; 2 g/cm²), fibronectin (FN, 5 g/cm²), fibrin (FB, 1.5 g/cm²), or left untreated (TCPS). For the FB coating, plates were first incubated overnight at 4° C. in fibrinogen (1 mg/mL), followed by three washes with 0.05% Tween 20 in phosphate-buffered saline (PBS) and 1 hour incubation with thrombin (0.6 mg/mL) at 37° C. All coatings were prepared in 50 mM bicarbonate coating buffer, pH 8.5, and rinsed three times with PBS before cell seeding. The amounts of adsorbed proteins were measured on separate plates using the bicinchoninic acid protein assay (Pierce, Rockford, Ill.) to verify adsorption of protein coatings.

MEK-1/2 Inhibition. VICs exposed to various concentrations and bioactivities of CBD were treated with U-0126 [1,4-diamino-2,3-dicyano-1,4-bis(2-aminophenylthio) butadiene; Calbiochem, San Diego, Calif.], PD-98059 (2-amino-3 methoxyflavone; 5 M; Calbiochem), or left untreated as a control to confirm the MAPK specificity of these inhibition experiments. U-0126 specifically inhibits MEK-1/2, thus inhibiting activation of ERK-1/2 (Favata et al., “Identification of a Novel Inhibitor of Mitogen-Activated Protein Kinase Kinase,” Journal of Biological Chemistry 273(29):18623-18632 (1998), which is hereby incorporated by reference in its entirety). PD-98059 is an alternate MEK inhibitor. Nine tissue samples were in each treatment group. These were the tissue samples used in subsequent analyses.

Quantification of Cell Number. At time points of 1, 3, and 5 days, VICs were lysed with radioimmunoprecipitation assay buffer [1% sodium deoxycholate, 0.1% SDS, 1% Triton X-100, 1 mM iodoacetamide, 140 mM NaCl, 10 mM Tris HCl, (pH 8.0)]. The amount of DNA in sample lysates was measured via the Quanti-iT PicoGreen assay (Invitrogen, Carlsbad, Calif.), according to the manufacturer's instructions.

Migration Assay. Migration was assayed via a modified fence method (Mann & West, 2002), wherein VICs were seeded within 2 mm² removable silicone wells, grown to confluency, and then allowed to migrate following the detachment of silicone isolators (defined as day 0). Grid-patterned transparencies were attached underneath plates containing VIC cultures to track cell movement over time. Photomicrographs were taken of the leading edge of cell migration under 40 magnification (Olympus IX51) every 24 hours for 5 days. Net cell edge displacement was measured by overlaying time course images and then quantifying migration distance (NIH ImageJ) by measuring the advancement of the leading cell edge subtracted from the migration area recorded on day 0 within a single grid space.

Apoptosis Assay. To ensure the health of the cell samples used in the calcification experiment, apoptosis was measured using an ELISA-based HT TiterTACS Assay Kit (Trevigen, Gaithersburg, Md.), which detects DNA fragmentation. At days 1 and 5, cells were fixed in 3.7% buffered formaldehyde solution for 7 minutes, washed with PBS, and postfixed in 100% methanol for 20 minutes. Following manufacturer's instructions, the cells were permeabilized with proteinase K, quenched with 2.5% H₂O₂ in methanol, and then incubated with the labeling reaction mix (TdT, Biotin-dNTP, unlabeled dNTP) to label breaks in DNA. Streptavidin-HRP and then TACS-Sapphire were added to the wells to detect apoptotic cells; the reaction was stopped with 2 N HCl, and absorbance was read at 450 nm.

RNA Isolation. Total RNA was isolated using TRI Reagent (Molecular Research Center, Cincinnati, Ohio), according to the manufacturer's instructions. VICs were lysed with 200 1 TRI Reagent per well at 4° C. with 50 protease inhibitor cocktail (BD Biosciences, San Jose, Calif.). The homogenate was stored at room temperature for 5 minutes to complete the dissociation of nucleoprotein complexes, at which point 0.15 mL chloroform per 600 1 TRI Reagent was added to the homogenate, followed by centrifugation at 13,000 g for 15 minutes. After centrifugation, RNA was precipitated from the upper aqueous phase by adding 0.3 mL isopropanol per 600 1 TRI Reagent to the tubes and then centrifuged at 13,000 g for 8 minutes. After this centrifugation step, the RNA pellet was washed with 75% ethanol and centrifuged at 8,000 g for 5 minutes. The RNA pellet was air dried and dissolved in 75 1 H₂O at 60° C. for 15 minutes. RNA samples were stored at 20° C. until subsequent use.

Quantitative Real-Time PCR Analysis. Custom primers for various markers of cell contractility and osteogenic activity were obtained from Invitrogen (Carlsbad, Calif.). For cDNA construction, 250 ng of original RNA isolated from samples were reverse transcribed using iScript (Bio-Rad Laboratories, Hercules, Calif.) as per manufacturer's instructions. Samples were processed for real-time PCR analysis by combining 0.5 1 of the cDNA construction, 5 M of primers, and SYBR Green SuperMix (Bio-Rad) in a 15-1 reaction, as specified in the manufacturer's protocol. For thermo cycling, a standard protocol was used: PCR reactions were run over 40 cycles of denaturing at 95° C. for 15 seconds and annealed at 60° C. for 1 minute; this was followed by a melting curve analysis for 80 cycles of 55° C. 0.5° C./cycle, 10 seconds per cycle, to further confirm the purity of the final PCR products, with each condition performed in triplicate (iCycler iQ Real-Time PCR Instrument, Bio-Rad). A standard comparative threshold cycle (or CT) method was used to analyze the PCR data. The CT of all samples were first normalized to -actin as an internal control, and then the CT values for experimental samples were further normalized to the negative control (VICs on Coll, which represented a non CBD condition).

Quantification of Nodule Number and Size. After 5 days of culture in the presence or absence of U-0126 or PD-98059, VIC cultures were stained with Alizarin Red S (ARS) to facilitate quantification of calcified nodules, as ARS stains mineralized deposits red. Cultures were fixed with 10% neutral buffered formalin, stored at 4° C. overnight, and stained with a 2% solution of ARS in PBS. Positively stained nodules were manually counted under a microscope (Olympus IX51 with Hamamatsu 285 digital camera and Simple PCI digital imaging software; Compix, Imaging Systems, Cranberry Township, Pa.). Nodule size was measured using ImageJ software (National Institutes of Health), and photomicrographs were captured under 40 and 100 magnifications.

CBD and ImmunAG Samples. 0.95 bioactivity CBD was isolated by HPLC from ImmunAG, a Humulus product of ImmunAG LLP. ImmunAG is a proprietary combination of CBD (39.5%), BCP (59.5%), and HMU (1%). The bioactivity of the BCP and HMU were not directly tested. They were likely approximately equal to the bioactivity of the CBD.

### Results

Multiplate wells containing calcifying VICs from 27 different tissue samples were treated with 5, 10, 25, 40, or 100 mg of either CBD or ImmunAG. Average calcification was computed using the number of nodules per well, and the average area per nodule. Mean nodule area for untreated wells was 3.27 mm² (SD=0.32, min=0.5, max=4.0) per well. All reported percent reductions in calcification were computed by dividing average nodule area of treated wells from 3.27 mm².

Means, standard deviations, and minimum and maximum values for CBD and ImmunAG for every tested mg concentration are provided in Table 8.

TABLE 8
Descriptive statisticis of calcification reduction
data of CBD and ImmunAG by mg concentration.

| mg | | CBD | ImmunAG |
|---|---|---|---|
| 5 | M | 19.42 | 24.32 |
| | SD | 1.8 | 1.77 |
| | Min | 15.4 | 20.1 |
| | Max | 22.1 | 27.8 |
| 10 | M | 25.56 | 30.39 |
| | SD | 3.04 | 4.97 |
| | Min | 18.5 | 19 |
| | Max | 32.5 | 39.7 |
| 25 | M | 32.69 | 38.53 |
| | SD | 4.33 | 4.44 |
| | Min | 26.3 | 26.9 |
| | Max | 42.6 | 46.1 |
| 40 | M | 42.98 | 52.83 |
| | SD | 4.8 | 3.65 |
| | Min | 35.8 | 44.6 |
| | Max | 54.5 | 60 |
| 100 | M | 55 | 64.21 |
| | SD | 6.66 | 7.91 |
| | Min | 41.9 | 51.3 |
| | Max | 66.1 | 78.1 |

Full data are provided in Table 9.

TABLE 9

| | Treatment | 5 mg | 10 mg | 25 mg | 40 mg | 100 mg |
|---|---|---|---|---|---|---|
| 1548 | CBD | 21.9 | 32.5 | 34.9 | 39.6 | 52.5 |
| 1730 | CBD | 19.2 | 18.5 | 27.7 | 43 | 43 |
| 1194 | CBD | 21.4 | 24.2 | 34 | 47.9 | 59.8 |
| 1785 | CBD | 21.2 | 27.9 | 29.2 | 42.6 | 55.6 |
| 1772 | CBD | 18.9 | 26.8 | 34.4 | 47.7 | 59 |
| 1169 | CBD | 19.7 | 24 | 30.3 | 46.8 | 50.7 |
| 1245 | CBD | 19.8 | 20.8 | 34.8 | 47.1 | 51.4 |
| 1496 | CBD | 19.4 | 31 | 31.6 | 44.8 | 41.9 |
| 1381 | CBD | 20.5 | 27.9 | 31.4 | 46.7 | 60.4 |
| 1380 | CBD | 16.7 | 25.4 | 42.4 | 36.5 | 62 |
| 1230 | CBD | 21.7 | 25.1 | 29.7 | 36.7 | 66.1 |
| 1655 | CBD | 18.8 | 27 | 42.6 | 43 | 59.6 |
| 1175 | CBD | 19.8 | 22.6 | 32.3 | 38.5 | 63.2 |
| 1617 | CBD | 17.6 | 28.1 | 35.6 | 51.1 | 51.5 |
| 1042 | CBD | 18.5 | 27.1 | 34.1 | 41.6 | 57 |
| 1640 | CBD | 18.8 | 22.3 | 31 | 46.1 | 64.7 |
| 1082 | CBD | 16.5 | 28.7 | 30.4 | 37.9 | 58.4 |
| 1743 | CBD | 18.5 | 24.3 | 29.7 | 36.9 | 56.7 |
| 1278 | CBD | 22.1 | 23.5 | 39.3 | 44.1 | 41.9 |
| 1134 | CBD | 20.4 | 25.7 | 34.3 | 42.1 | 51.9 |
| 1452 | CBD | 16.6 | 26.3 | 38.9 | 39.2 | 43.6 |
| 1696 | CBD | 19.7 | 24.8 | 28.6 | 35.8 | 56.6 |
| 1765 | CBD | 18.7 | 27.2 | 32.2 | 39.6 | 56.3 |
| 1032 | CBD | 21.1 | 27.4 | 31.9 | 38.9 | 59.3 |
| 1850 | CBD | 21.9 | 21.3 | 26.5 | 44.1 | 52 |
| 2018 | CBD | 15.4 | 25.6 | 28.6 | 47.6 | 53.6 |
| 1205 | CBD | 19.6 | 24.1 | 26.3 | 54.5 | 56.4 |
| 1548 | ImmunAG | 23.7 | 23.4 | 41.9 | 46.8 | 56.1 |
| 1730 | ImmunAG | 21.3 | 23 | 26.9 | 54.8 | 74.5 |
| 1194 | ImmunAG | 24.4 | 26.3 | 39.2 | 51.6 | 78.1 |
| 1785 | ImmunAG | 23.3 | 30.6 | 37.6 | 54.3 | 73.4 |
| 1772 | ImmunAG | 23.8 | 39.7 | 38.4 | 49.8 | 53.4 |
| 1169 | ImmunAG | 25 | 36.1 | 38.1 | 49.9 | 53.7 |
| 1245 | ImmunAG | 25.3 | 29.2 | 38.9 | 52.9 | 59.1 |
| 1496 | ImmunAG | 21.3 | 29.2 | 46.1 | 57 | 66.8 |
| 1381 | ImmunAG | 24.2 | 23.5 | 38.1 | 55.7 | 54.9 |
| 1380 | ImmunAG | 23.4 | 31.5 | 35.1 | 56.6 | 51.3 |
| 1230 | ImmunAG | 26.5 | 32.3 | 45.9 | 55.3 | 66.6 |
| 1655 | ImmunAG | 25.4 | 32 | 41.4 | 50.2 | 57.3 |
| 1175 | ImmunAG | 23.7 | 34.7 | 39.6 | 53.3 | 56.7 |
| 1617 | ImmunAG | 24.6 | 31.2 | 35.7 | 51.8 | 56.1 |
| 1042 | ImmunAG | 24.2 | 19 | 42.6 | 53 | 63.5 |
| 1640 | ImmunAG | 22.9 | 25.1 | 42.5 | 53.1 | 72.4 |
| 1082 | ImmunAG | 27.5 | 31.3 | 36.5 | 52 | 70 |
| 1743 | ImmunAG | 24.7 | 27.8 | 42.7 | 50.7 | 58.8 |
| 1278 | ImmunAG | 25.1 | 33.5 | 31.9 | 49.7 | 74.5 |
| 1134 | ImmunAG | 27.8 | 37.7 | 39.1 | 57.2 | 64.3 |
| 1452 | ImmunAG | 25.3 | 34.1 | 43.3 | 60 | 77.7 |
| 1696 | ImmunAG | 20.1 | 35.4 | 38.3 | 54.1 | 64.6 |
| 1765 | ImmunAG | 25.1 | 33 | 39.6 | 59.9 | 71.6 |
| 1032 | ImmunAG | 22.2 | 27.2 | 34.3 | 48 | 62.8 |
| 1850 | ImmunAG | 26.2 | 30 | 29.5 | 44.6 | 63.3 |
| 2018 | ImmunAG | 24.7 | 27.5 | 37.3 | 51.7 | 67.7 |
| 1205 | ImmunAG | 24.9 | 36.1 | 39.7 | 52.4 | 64.5 |

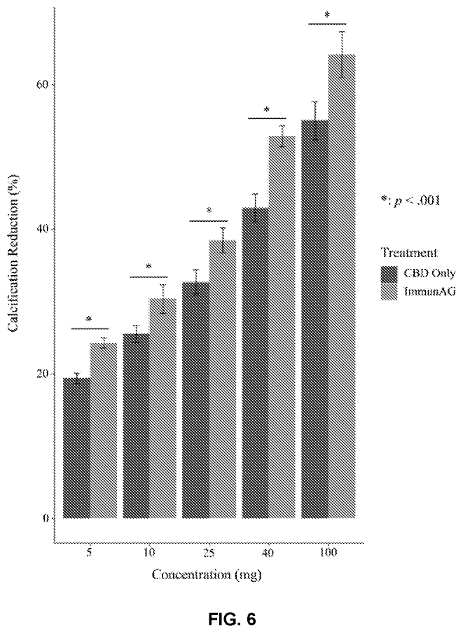

Paired-samples t-tests determined that ImmunAG reduced calcification significantly more than CBD alone at every mg concentration: At 5 mg, t(26)=9.87,p<0.001, d=1.90; at 10 mg, t(26)=4.20,p<0.001, d=0.81; at 25 mg, t(26)=5.23, p<0.001, d=1.00; at 40 mg, t(26)=7.76, p<0.001, d=1.49; and at 100 mg, t(26) =4.05,p<0.001, d=0.78. A barplot of observed differences is given in FIG. 6.

### Conclusion

This study demonstrated that a combination of high bioactivity CBD, BCP, and HMU reduced VIC calcification more than high bioactivity CBD alone. It remains to be seen whether possible synergistic effects afforded by this combination of compounds extends beyond VIC calcification. CBD has been shown to have anxiolytic, antidepressant, antipsychotic, anticonvulsant, anti-nausea, antioxidant, antiinflammatory, anti-arthritic, and anti-neoplastic properties (Ligresti et al., “From Phytocannabinoids to Cannabinoid Receptors and Endocannabinoids: Pleiotropic Physiological and Pathological Roles through Complex Pharmacology,” Physiological Reviews 96(4):1593-1659 (2016), which is hereby incorporated by reference in its entirety). BCP has shown promising for anti-endemic, anti-tumoral, anti-oxidant, anti-microbial, and anti-inflammatory properties (Dahham et al., “The Anticancer, Antioxidant and Antimicrobial Properties of the Sesquiterpene β-Caryophyllene from the Essential Oil of Aquilaria crassna,” Molecules 20(7):11808-29 (2015), which is hereby incorporated by reference in its entirety). As phytoceutical approaches to medicine continue to gain traction, a uncovering the ways that each of these properties interact will constitute an exciting new frontier for science.

### Example 5—Liver Cancer Study with ImmunAG

### Materials and Methods for Example 5

Patient Characteristics. There were 99 men and 18 women in the trial. The average age was 53, with a minimum age of 31 and a maximum age of 78. Eighty-six patients (73.5%) were preoperative TACE negative, and 31 patients (26.49%) were preoperative TACE positive. Seventeen patient (14.52%) were HBsAg negative and 100 patients (85.47%) were HBsAg positive. One hundred six patients (95.49%) were Anti-HCV negative and 11 patients (4.5%) were Anti-HCV positive. Eighteen patients (16.1%) were negative for cirrhosis and 99 patients (83.9%) were positive for cirrhosis. Preoperative alpha-fetoprotein (μg/L) was an average of 18.3 with a minimum value of 1.7 and a maximum value of 1089. Cumulative tumor size was an average of 4.89 cm with a minimum of 2.37 cm and a maximum value of 19.62 cm. Fifty-three people had 2 tumors in the liver, 52 people has 3 liver tumors and 12 people had 4 tumors. The average tumor number is 3 N. There were 7 cases (5.98%) of high Necrosis differentiation, 106 cases of medium (90.59%) Necrosis differentiation, and 4 cases (3.41%) of low Necrosis differentiation. Only 18 patient (15%) did not have liver capsule invasion. The rest, 99 patient (84.6%) had liver capsule invasion in some form.

All these patients were suffering from Standard Risk (SR) Hepatoblastoma- and not Hepatocellular carcinoma or Cholangiocarcinoma. Of these patients, 57 of the men, and 7 of the women, had surgical resectioning of some degree. However at this point of treatment resectioning was not considered a viable option.

There were 54 men and 10 women in the Cisplatin alone treatment group. There were 45 men and 8 women in the Cisplatin+ImmunAG group. The Cisplatin alone treatment group received a dosage of 50-70 mg/m², every four weeks for a duration of the study. The Cisplatin+ImmunAG group received the same dosage of Cisplatin as noted above in combination with a 80 mg-600 mg/day dose of ImmunAG (composition comprising CBD, humulene, and β-caryophyllene). The exact dose of ImmunAG was determined based on patient's sex and height, and was administered in three doses over the course of the day.

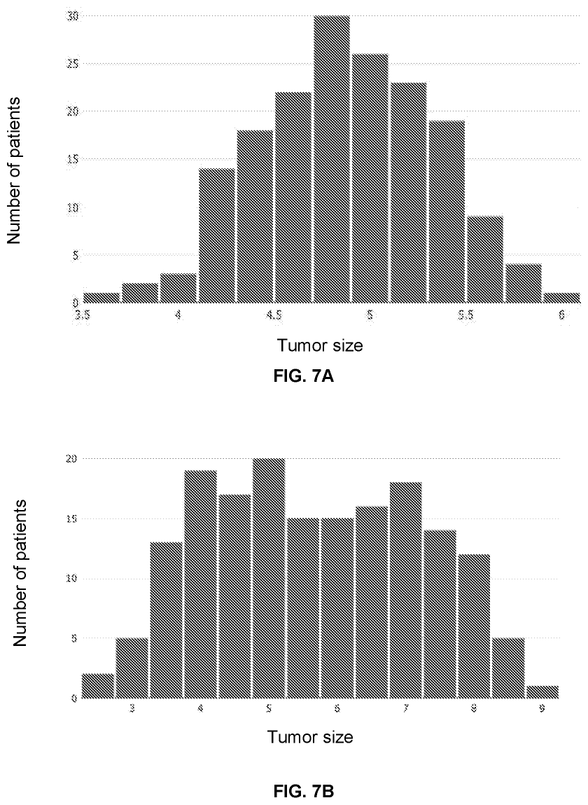

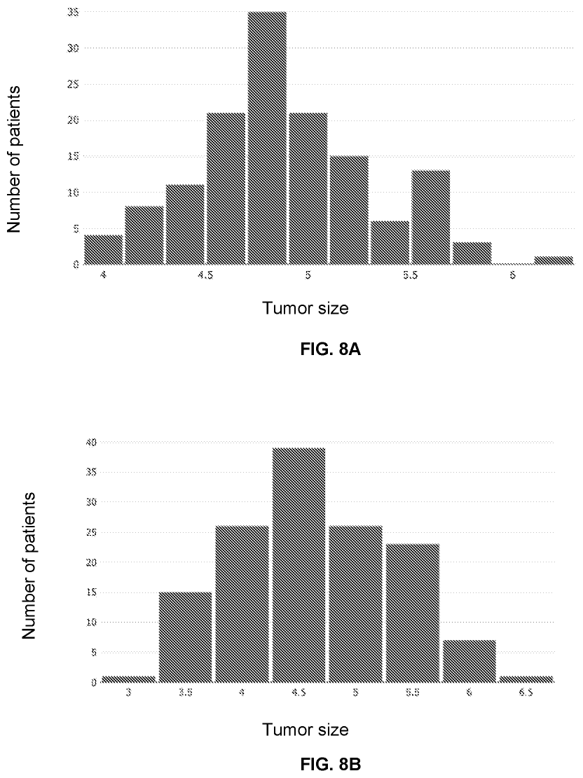

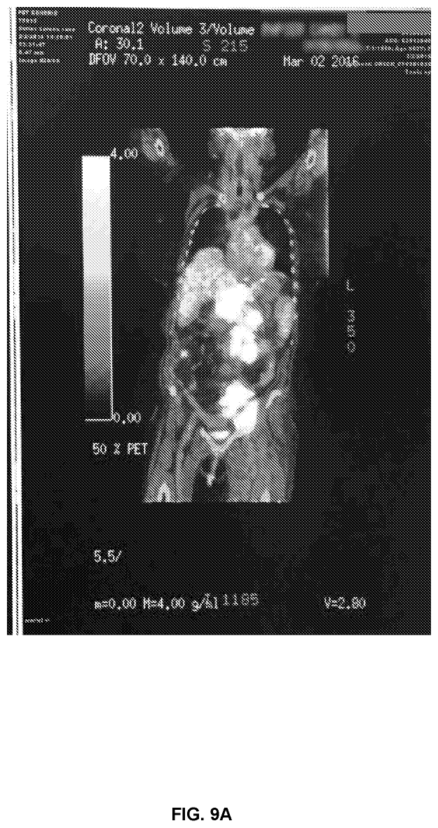

The mean tumors decreased (−5%) to 0.952 from 1 for “Cisplatin+ImmunAG adjuvant treatment” (see FIGS. 8A-8B) and increased on the average from 1 to 1.17 (17%) for Cisplatin alone treatment (see FIGS. 7A-7B). With 90% confidence interval it can be predicted that Cisplatin with ImmunAG reduced liver cancer tumors 22% more than Cisplatin alone.

### Example 6—Case Study with ImmunAG

### Materials and Methods for Example 6

Patient information. Jane Doe, 62 year-old postmenopausal woman of Caucasian decent from San Jose, Calif. In 2013, she found a lump in her right breast. She was diagnosed with infiltrating ductal adenocarcinoma; 6.1 cm lesion in lower quadrant of right breast which were FNA+ for adenocarcinoma. The tumor was determined to be HER2-negative/ER+/PR+ tumor. Bone scan and chest diagnostic CT revealed no metastases. Patient was treated with Neoadjuvant therapy with dose-dense AC: doxorubicin 60 mg/m² IV q2 weeks followed by weekly paclitaxel 80 mg/m²×12. Patient underwent breast-conserving therapy (malignant cells in 7 axillary lymph nodes). Surgery was performed followed by chest wall and regional lymph node radiation therapy (5×/week for 6 weeks). Patient started nonsteroidal aromatase inhibitor.

Five months after adjuvant chemotherapy treatment, patient complained of bone pain. Bone scan and CT scan revealed several metastases: 2 lesions on left humeral bone measuring 2-3 cm, 2 lesions on liver measuring 2-3 cm; 1 lesion on lung measuring 2-3 cm. Patient identified as potentially resistant to taxane (having progressed within 12 months of last adjuvant therapy). Liver biopsy and pathology showed metastases consistent with original breast cancer. Patient diagnosed with stage IV cancer. Histology confirmed HER2-negative/ER+/PR+ disease. Patient began therapy with denosumab for bone metastases. Patient also received Xeloda 1000 mg/m² PO BID day 1-14. Patient decided to discontinue chemotherapy.

Her son procured ImmunAG. She was given 500 mg of ImmunAG per day. 200 mg at 8 am, 100 mg at 2 pm and 200 mg at 8 pm.

### Results

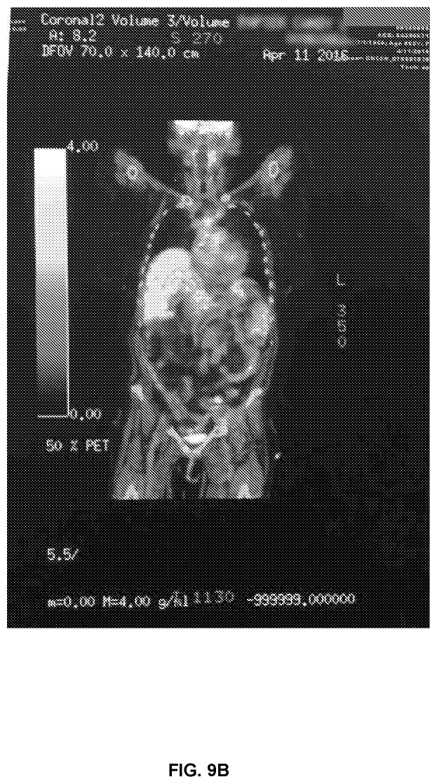

As can be seen in the PET chart on Apr. 11, 2016 (FIG. 9B compared to FIG. 9A), 95% of the metastasized cancer had disappeared in 39 days. ImmunAG acts as a “Check Point Protein” inhibitor. Check Point Proteins allow the cancer cells to hide from the immune system. Because of the interference of the check point proteins by ImmunAG, the immune system is able to recognize the cancer cells and attacks them. The immune system is ubiquitous and in this case is able to attack the metastasized cancer cells

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A Humulus plant having a cannabinoid concentration in its inflorescence of at least 75 mg/g (dry weight).

2. The Humulus plant of claim 1, having a cannabinoid concentration in its leaves of at least 15 mg/g (dry weight).

3. The Humulus plant of claim 1, wherein the cannabinoid concentration in the inflorescence is at least 100 mg/g (dry weight).

4. The Humulus plant of claim 2, wherein the cannabinoid concentration in the leaves is at least 20 mg/g (dry weight).

5. The Humulus plant of claim 1, wherein the plant is a Humulus yunnanensis plant.

6. The Humulus plant of claim 1, wherein said plant contains one or more cannabinoids selected from the group consisting of Cannabigerol (CBG), Cannabichromene (CBC), Cannabidiol (CBD), Cannabielsoin (CBE) and Cannabidivarin (CBDV).

7. The Humulus plant of claim 1, wherein the plant does not contain tetrahydrocannabinolic acid (THCA) or tetrahydrocannabinol (THC).

8. An extract of the plant according to claim 1.

9. The extract of claim 8, wherein said extract comprises concentrated levels of humulene, β-caryophyllene, and cannabidiol.

10. The extract of claim 9, wherein the extract comprises 1.5% humulene, 60% β-caryophyllene, and 38.5% cannabidiol.

11. A composition comprising the extract of claim 8.

12. A composition comprising:
1%-10% humulene;
5%-90% β-caryophyllene; and
5%-90% cannabidiol.

13. The composition of claim 12 comprising:
1.5% humulene;
60% β-caryophyllene; and
38.5% cannabidiol.

14. The composition of claim 13 consisting essentially of:
1.5% humulene;
60% β-caryophyllene; and
38.5% cannabidiol.

15. The composition of claim 11, wherein said composition does not contain tetrahydrocannabinolic acid (THCA) or tetrahydrocannabinol (THC).

16. The composition of claim 11, wherein the composition is formulated for oral administration.

17. The composition of claim 16, wherein the composition is formulated as a capsule, tablet, liquid, powder, granules, suspensions, or oil.

18. The composition of claim 16, wherein the composition is formulated as a time-release tablet.

19. The composition of claim 11, wherein the composition is formulated for topical administration.

20. The composition of claim 19, wherein the composition is formulated as a cream, lotion, emulsion, powder, paste, gel, spray, ointment, solution, foam, and oil.

21. A method of modulating endocannabinoid system activity in a subject, said method comprising:
selecting a subject in need of endocannabinoid system modulation, and
administering to said selected subject, the composition of claim 11 15 in an amount effective to modulate endocannabinoid system activity in the subject.

22.-40. (canceled)
